SEQUENCE LISTING (6 sequences)

SEQ ID NO: 1 - DNA, 1731 bp, Artificial Sequence
  Synthetic spider silk protein
atggctagcatgactggtggacagcaaatgggtcgcggatccatgggttctagcgcggctgcagccgcggcagctgcgtccggcccgggtggctacggtccggaaaaccagggtccatctggcccgggtggctacggtcctggcggtccgggttctagcgcggctgcagccgcggcagctgcgtccggcccgggtggctacggtccggaaaaccagggtccatctggcccgggtggctacggtcctggcggtccgggttctagcgcggctgcagccgcggcagctgcgtccggcccgggtggctacggtccggaaaaccagggtccatctggcccgggtggctacggtcctggcggtccgggttctagcgcggctgcagccgcggcagctgcgtccggcccgggtggctacggtccggaaaaccagggtccatctggcccgggtggctacggtcctggcggtccgggttctagcgcggctgcagccgcggcagctgcgtccggcccgggtggctacggtccggaaaaccagggtccatctggcccgggtggctacggtcctggcggtccgggttctagcgcggctgcagccgcggcagctgcgtccggcccgggtggctacggtccggaaaaccagggtccatctggcccgggtggctacggtcctggcggtccgggttctagcgcggctgcagccgcggcagctgcgtccggcccgggtggctacggtccggaaaaccagggtccatctggcccgggtggctacggtcctggcggtccgggttctagcgcggctgcagccgcggcagctgcgtccggcccgggtggctacggtccggaaaaccagggtccatctggcccgggtggctacggtcctggcggtccgggttctagcgcggctgcagccgcggcagctgcgtccggcccgggtggctacggtccggaaaaccagggtccatctggcccgggtggctacggtcctggcggtccgggttctagcgcggctgcagccgcggcagctgcgtccggcccgggtggctacggtccggaaaaccagggtccatctggcccgggtggctacggtcctggcggtccgggttctagcgcggctgcagccgcggcagctgcgtccggcccgggtggctacggtccggaaaaccagggtccatctggcccgggtggctacggtcctggcggtccgggttctagcgcggctgcagccgcggcagctgcgtccggcccgggtggctacggtccggaaaaccagggtccatctggcccgggtggctacggtcctggcggtccgggttctagcgcggctgcagccgcggcagctgcgtccggcccgggtggctacggtccggaaaaccagggtccatctggcccgggtggctacggtcctggcggtccgggttctagcgcggctgcagccgcggcagctgcgtccggcccgggtggctacggtccggaaaaccagggtccatctggcccgggtggctacggtcctggcggtccgggttctagcgcggctgcagccgcggcagctgcgtccggcccgggtggctacggtccggaaaaccagggtccatctggcccgggtggctacggtcctggcggtccgggttctagcgcggctgcagccgcggcagctgcgtccggcccgggtggctacggtccggaaaaccagggtccatctggcccgggtggctacggtcctggcggtccgggctaa

SEQ ID NO: 2 - protein, 576 aa, Artificial Sequence
  Synthetic Construct
MASMTGGQQMGRGSMGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPG

SEQ ID NO: 3 - DNA, 1683 bp, Artificial Sequence
  R16 silk protein
atggctagcatgactggtggacagcaaatgggtcgcggatccatgggcccgggttctagcgcggctgcagccgcggcagctgcgtccggcccgggtcagggccagggtcagggtcaaggccagggtggccgtccttctgacacctacggcccgggttctagcgcggctgcagccgcggcagctgcgtccggcccgggtcagggccagggtcagggtcaaggccagggtggccgtccttctgacacctacggcccgggttctagcgcggctgcagccgcggcagctgcgtccggcccgggtcagggccagggtcagggtcaaggccagggtggccgtccttctgacacctacggcccgggttctagcgcggctgcagccgcggcagctgcgtccggcccgggtcagggccagggtcagggtcaaggccagggtggccgtccttctgacacctacggcccgggttctagcgcggctgcagccgcggcagctgcgtccggcccgggtcagggccagggtcagggtcaaggccagggtggccgtccttctgacacctacggcccgggttctagcgcggctgcagccgcggcagctgcgtccggcccgggtcagggccagggtcagggtcaaggccagggtggccgtccttctgacacctacggcccgggttctagcgcggctgcagccgcggcagctgcgtccggcccgggtcagggccagggtcagggtcaaggccagggtggccgtccttctgacacctacggcccgggttctagcgcggctgcagccgcggcagctgcgtccggcccgggtcagggccagggtcagggtcaaggccagggtggccgtccttctgacacctacggcccgggttctagcgcggctgcagccgcggcagctgcgtccggcccgggtcagggccagggtcagggtcaaggccagggtggccgtccttctgacacctacggcccgggttctagcgcggctgcagccgcggcagctgcgtccggcccgggtcagggccagggtcagggtcaaggccagggtggccgtccttctgacacctacggcccgggttctagcgcggctgcagccgcggcagctgcgtccggcccgggtcagggccagggtcagggtcaaggccagggtggccgtccttctgacacctacggcccgggttctagcgcggctgcagccgcggcagctgcgtccggcccgggtcagggccagggtcagggtcaaggccagggtggccgtccttctgacacctacggcccgggttctagcgcggctgcagccgcggcagctgcgtccggcccgggtcagggccagggtcagggtcaaggccagggtggccgtccttctgacacctacggcccgggttctagcgcggctgcagccgcggcagctgcgtccggcccgggtcagggccagggtcagggtcaaggccagggtggccgtccttctgacacctacggcccgggttctagcgcggctgcagccgcggcagctgcgtccggcccgggtcagggccagggtcagggtcaaggccagggtggccgtccttctgacacctacggcccgggttctagcgcggctgcagccgcggcagctgcgtccggcccgggtcagggccagggtcagggtcaaggccagggtggccgtccttctgacacctacggctaa

SEQ ID NO: 4 - protein, 560 aa, Artificial Sequence
  Synthetic Construct
MASMTGGQQMGRGSMGPGSSAAAAAAAASGPGQGQGQGQGQGGRPSDTYGPGSSAAAAAAAASGPGQGQGQGQGQGGRPSDTYGPGSSAAAAAAAASGPGQGQGQGQGQGGRPSDTYGPGSSAAAAAAAASGPGQGQGQGQGQGGRPSDTYGPGSSAAAAAAAASGPGQGQGQGQGQGGRPSDTYGPGSSAAAAAAAASGPGQGQGQGQGQGGRPSDTYGPGSSAAAAAAAASGPGQGQGQGQGQGGRPSDTYGPGSSAAAAAAAASGPGQGQGQGQGQGGRPSDTYGPGSSAAAAAAAASGPGQGQGQGQGQGGRPSDTYGPGSSAAAAAAAASGPGQGQGQGQGQGGRPSDTYGPGSSAAAAAAAASGPGQGQGQGQGQGGRPSDTYGPGSSAAAAAAAASGPGQGQGQGQGQGGRPSDTYGPGSSAAAAAAAASGPGQGQGQGQGQGGRPSDTYGPGSSAAAAAAAASGPGQGQGQGQGQGGRPSDTYGPGSSAAAAAAAASGPGQGQGQGQGQGGRPSDTYGPGSSAAAAAAAASGPGQGQGQGQGQGGRPSDTYG

SEQ ID NO: 5 - DNA, 1923 bp, Artificial Sequence
  S16 silk protein
atggctagcatgactggtggacagcaaatgggtcgcggatccatgggttctgcggctgcagccgcggcagctgcgggtccgggcggtggcaacggtggccgtccgtctgacacctacggtgcgccgggtggcggtaacggtggccgtccttcttcctcttacggttctgcggctgcagccgcggcagctgcgggtccgggcggtggcaacggtggccgtccgtctgacacctacggtgcgccgggtggcggtaacggtggccgtccttcttcctcttacggttctgcggctgcagccgcggcagctgcgggtccgggcggtggcaacggtggccgtccgtctgacacctacggtgcgccgggtggcggtaacggtggccgtccttcttcctcttacggttctgcggctgcagccgcggcagctgcgggtccgggcggtggcaacggtggccgtccgtctgacacctacggtgcgccgggtggcggtaacggtggccgtccttcttcctcttacggttctgcggctgcagccgcggcagctgcgggtccgggcggtggcaacggtggccgtccgtctgacacctacggtgcgccgggtggcggtaacggtggccgtccttcttcctcttacggttctgcggctgcagccgcggcagctgcgggtccgggcggtggcaacggtggccgtccgtctgacacctacggtgcgccgggtggcggtaacggtggccgtccttcttcctcttacggttctgcggctgcagccgcggcagctgcgggtccgggcggtggcaacggtggccgtccgtctgacacctacggtgcgccgggtggcggtaacggtggccgtccttcttcctcttacggttctgcggctgcagccgcggcagctgcgggtccgggcggtggcaacggtggccgtccgtctgacacctacggtgcgccgggtggcggtaacggtggccgtccttcttcctcttacggttctgcggctgcagccgcggcagctgcgggtccgggcggtggcaacggtggccgtccgtctgacacctacggtgcgccgggtggcggtaacggtggccgtccttcttcctcttacggttctgcggctgcagccgcggcagctgcgggtccgggcggtggcaacggtggccgtccgtctgacacctacggtgcgccgggtggcggtaacggtggccgtccttcttcctcttacggttctgcggctgcagccgcggcagctgcgggtccgggcggtggcaacggtggccgtccgtctgacacctacggtgcgccgggtggcggtaacggtggccgtccttcttcctcttacggttctgcggctgcagccgcggcagctgcgggtccgggcggtggcaacggtggccgtccgtctgacacctacggtgcgccgggtggcggtaacggtggccgtccttcttcctcttacggttctgcggctgcagccgcggcagctgcgggtccgggcggtggcaacggtggccgtccgtctgacacctacggtgcgccgggtggcggtaacggtggccgtccttcttcctcttacggttctgcggctgcagccgcggcagctgcgggtccgggcggtggcaacggtggccgtccgtctgacacctacggtgcgccgggtggcggtaacggtggccgtccttcttcctcttacggttctgcggctgcagccgcggcagctgcgggtccgggcggtggcaacggtggccgtccgtctgacacctacggtgcgccgggtggcggtaacggtggccgtccttcttcctcttacggttctgcggctgcagccgcggcagctgcgggtccgggcggtggcaacggtggccgtccgtctgacacctacggtgcgccgggtggcggtaacggtggccgtccttcttcctcttacggctaa

SEQ ID NO: 6 - protein, 640 aa, Artificial Sequence
  Synthetic Construct
MASMTGGQQMGRGSMGSAAAAAAAAGPGGGNGGRPSDTYGAPGGGNGGRPSSSYGSAAAAAAAAGPGGGNGGRPSDTYGAPGGGNGGRPSSSYGSAAAAAAAAGPGGGNGGRPSDTYGAPGGGNGGRPSSSYGSAAAAAAAAGPGGGNGGRPSDTYGAPGGGNGGRPSSSYGSAAAAAAAAGPGGGNGGRPSDTYGAPGGGNGGRPSSSYGSAAAAAAAAGPGGGNGGRPSDTYGAPGGGNGGRPSSSYGSAAAAAAAAGPGGGNGGRPSDTYGAPGGGNGGRPSSSYGSAAAAAAAAGPGGGNGGRPSDTYGAPGGGNGGRPSSSYGSAAAAAAAAGPGGGNGGRPSDTYGAPGGGNGGRPSSSYGSAAAAAAAAGPGGGNGGRPSDTYGAPGGGNGGRPSSSYGSAAAAAAAAGPGGGNGGRPSDTYGAPGGGNGGRPSSSYGSAAAAAAAAGPGGGNGGRPSDTYGAPGGGNGGRPSSSYGSAAAAAAAAGPGGGNGGRPSDTYGAPGGGNGGRPSSSYGSAAAAAAAAGPGGGNGGRPSDTYGAPGGGNGGRPSSSYGSAAAAAAAAGPGGGNGGRPSDTYGAPGGGNGGRPSSSYGSAAAAAAAAGPGGGNGGRPSDTYGAPGGGNGGRPSSSYG

(19) United States
(12) Patent Application Publication
(10) Pub. No.: US 2011/0129510 A1
(43) Pub. Date: Jun. 2, 2011

(54) FIBROUS SURFACE STRUCTURE CONTAINING ACTIVE INGREDIENTS WITH CONTROLLED RELEASE OF ACTIVE INGREDIENTS, USE THEREOF AND METHOD FOR THE PRODUCTION THEREOF

(75) Inventors: Burghard Liebmann, Bensheim (DE); Evgueni Klimov, Ludwigshafen (DE)

(73) Assignee: BASF SE, Ludwigshafen (DE)

(21) Appl. No.: 13/058,033

(22) PCT Filed: Aug. 7, 2009
(86) PCT No.: PCT/EP09/60293

§ 371 Date: Feb. 8, 2011

(30) Foreign Application Priority Data
Aug. 8, 2008 (EP) 08162122.9

(51) Int. Cl. A61K 9/70; A61K 47/32; A61K 47/30; A61K 8/72; A01N 25/00; A61K 8/02; A01P 3/00; A23P 1/10; A23L 1/308; A23K 1/16

(52) U.S. Cl. 424/401; 514/772.4; 514/772.3; 514/772.2; 514/772.5; 424/443; 426/648; 426/442; 426/515

(57) ABSTRACT

The invention relates to a fibrous surface structure containing active ingredients with an adjustable active ingredient release profile, comprising a fibrous, polymeric, soluble and/or degradable active ingredient substrate and at least one active ingredient that is associated with the substrate and can be released by the fibrous surface structure; to formulations containing active ingredients, comprising such fibrous surface structures; to the use of fibrous surface structures containing active ingredients according to the invention for producing formulations containing active ingredients; and to a method for producing fibrous surface structures according to the invention.

The invention relates to active ingredient-containing fibrous sheetlike structures with an adjustable active ingredient release profile, comprising a fibrous, polymeric, soluble and/or degradable active ingredient carrier and at least one active ingredient which is associated with the carrier and can be released by the fibrous sheetlike structure; to active ingredient-containing formulations comprising such fibrous sheetlike structures; to the use of inventive active ingredient-containing fibrous sheetlike structures for production of active ingredient-containing formulations; and to processes for production of inventive fibrous sheetlike structures.

### STATE OF THE ART

For production of nano- and mesofibers, the person skilled in the art is aware of a multitude of processes, among which electrospinning is currently of the greatest significance. In this process, which is described, for example, by D. H. Reneker, H. D. Chun in Nanotechn. 7 (1996), pages 216 ff., a polymer melt or a polymer solution is typically exposed to a high electrical field at an edge which serves as an electrode. This can be achieved, for example, by extruding the polymer melt or polymer solution through a cannula connected to one pole of a voltage source in an electrical field under low pressure. Owing to the resulting electrostatic charging of the polymer melt or polymer solution, the result is a material flow directed toward the counterelectrode, which solidifies on the way to the counterelectrode. Depending on the electrode geometries, this process affords nonwovens or assemblies of ordered fibers.

DE-A1-10133393 discloses a process for producing hollow fibers with an internal diameter of 1 to 100 nm, in which a solution of a water-insoluble polymer—for example a poly-L-lactide solution in dichloromethane or a nylon-46 solution in pyridine—is electrospun. A similar process is also known from WO-A1-01/09414 and DE-A1-10355665.

DE-A1-19600162 discloses a process for producing lawnmower wire or textile sheetlike structures, in which polyamide, polyester or polypropylene as a thread-forming polymer, a maleic anhydride-modified polyethylene/polypropylene rubber and one or more aging stabilizers are combined, melted and mixed with one another, before this melt is melt-spun.

DE-A1-10 2004 009 887 relates to a process for producing fibers having a diameter of <50 μm by electrostatic spinning or spraying of a melt of at least one thermoplastic polymer.

A detailed description of electrospinning processes, properties of fibers and the possible uses thereof can be found in A. Greiner and J. Wendorff, Angew. Chemie Int. Ed., 2007, 119, 5770-5805.

A further suitable process for producing fiber nonwovens is known as centrifuge spinning (also called rotor spinning). EP 624 665 B1 and EP 1 088 918 A1 (both BASF applications) disclose a process for producing fibrous structures from melamine-formaldehyde resin and blends thereof with thermoplastic polymers by means of centrifugal spinning processes on a spinning plate.

A process and a device for production of fibers from the melt of different polymer materials with the aid of centrifugal forces are also described in DE 10 2005 048 939 A1.

WO-A-2001/54667 and WO-A-2004/014304 disclose amorphous pharmaceutical formulations and processes for production thereof. By electrospinning polymer-active pharmaceutical ingredient solutions, it was possible to obtain stable amorphous formulations. However, no specific information is given about active ingredient release and the control thereof.

WO-A-2007/082936 describes the use of amphiphilic, self-assembly proteins for formulation of sparingly water-soluble effect substances by dispersing the effect substances in a protein-containing protective colloid. After mixing the sparingly water-soluble effect substances and the amphiphilic, self-assembly proteins in a common disperse phase, and subsequent phase separation into a high-protein and -effect substance phase and a low-protein and -effect substance phase, protein microbeads are present, into which the sparingly water-soluble effect substances have been encapsulated.

WO-A-2007/093232 describes nanoparticulate formulations of active crop protection ingredients, in which the nanoparticles have core-shell structures with a mean particle diameter of 0.05 to 2.0 μm and the active crop protection ingredient is present in the core in x-ray-amorphous form together with one or more polymers, wherein the polymer is insoluble or only partly soluble in water or aqueous solutions or water-solvent mixtures, and the shell consists of a stabilizing shell matrix. These formulations are producible by a process which comprises (a) producing a solution of the active crop protection ingredient in a water-immiscible organic solvent, (b) dissolving the core polymer in a water-immiscible organic solvent; and emulsifying the mixture resulting from (a) and (b) with an aqueous solution comprising components of the shell matrix by injecting the corresponding solutions into a mixing chamber and removing the organic solvent after the emulsification.

### SUMMARY OF THE INVENTION

The polymer-based formulations of active ingredients and effect substances known to date are still afflicted with disadvantages. Especially the control of the release of the formulated active ingredients, for example over prolonged periods, constitutes a problem which is yet to be solved satisfactorily to date.

The inclusion of agrochemical, pharmaceutical and cosmetic effect substances in synthetic polymers or in polymer mixtures within the temperature range of 5-90° C. under standard pressure from a dilute polymer-active ingredient solution would be particularly advantageous for sparingly soluble and thermally sensitive effect substances. Of particular value would be the use of biodegradable or biocompatible polymers.

It was therefore an object of the present invention to provide a process which allows the formulation of such active ingredients using polymer materials as formulating aids, while enabling better adjustment of the active ingredient release than is known from the prior art.

The terms “active ingredients” and “effect substances” are used synonymously hereinafter.

### DESCRIPTION OF FIGURES

The appended figures show:

FIG. 1A SEM images of PVP polymer fibers, obtainable by electrospinning PVP polymer solutions with different contents of the epoxiconazole active ingredient.

FIG. 1B the recovery rates for the epoxiconazole active ingredient in different PVP matrices produced in accordance with the invention (fiber nonwovens 1 and 2) compared to corresponding calibration samples.

FIG. 2 the results of WAXS analyses on freshly produced fibrous sheetlike structures composed of PVP-epoxiconazole at contents of 9, 23 and 33% by weight of epoxiconazole compared to pure PVP or crystalline epoxiconazole.

FIG. 3 the results of WAXS analyses on fibrous sheetlike structures produced in accordance with the invention from PVP-epoxiconazole which have been stored at different temperatures, compared to pure PVP or crystalline epoxiconazole; for this purpose, each sample was stored at +40° C., -10° C. and 0° C. for 24 hours in each case, and then at 20° C. for 72 h.

FIG. 4A SEM images of PVP-β-carotene fibers produced in accordance with the invention with different β-carotene contents.

FIG. 4B the recovery rates for the β-carotene active ingredient in different PVP matrices produced in accordance with the invention (fiber nonwovens 1 and 2) compared to corresponding calibration samples.

FIG. 5 the results of WAXS analyses on fibrous sheetlike structures produced in accordance with the invention from PVP-β-carotene compared to pure PVP or crystalline β-carotene.

FIG. 6 the results of WAXS analyses on fibrous sheetlike structures produced in accordance with the invention from PVP-β-carotene which have been stored at different temperatures, compared to pure PVP or crystalline β-carotene; for this purpose, each sample was stored at +40° C., -10° C. and 0° C. for 24 hours in each case, and then at 20° C. for 72 h.

FIG. 7 SEM images of PMMA-epoxiconazole fibers produced in accordance with the invention with different epoxiconazole contents.

FIG. 8 the results of the WAXS analyses on fibrous sheetlike structures formed from PMMA-epoxiconazole at different epoxiconazole contents.

FIG. 9 the respective release profiles of epoxiconazole from the biodegradable polyester Ecoflex® as a film or fibrous sheetlike structure.

FIG. 10 the different release profiles of epoxiconazole from biodegradable polyester Ecoflex®, PVP and PMMA.

FIG. 11 the different release profiles of epoxiconazole from fibrous sheetlike structures, produced from PVP, PMMA and 1:1 or 1:5 blends of PVP and PMMA.

FIG. 12 microscope images of cross sections through active ingredient-free fibers of PMMA and PVP;

FIG. 13 the different release profiles of epoxiconazole from fibrous sheetlike structures produced from PVP, Ecoflex® and a 1:1 blend of PVP and Ecoflex;

FIG. 14 electron microscopy (SEM) images of sheetlike C16 spider silk protein structures (fibers) with incorporated clotrimazole active ingredient;

FIG. 15 crystallinity studies (WAXS in transmission) of the clotrimazole active ingredient in the C16 spider silk protein formulations obtained by electrospinning compared to pure clotrimazole;

FIG. 16 the release of the clotrimazole active ingredient from a C16 spider silk protein formulation obtained by electrospinning and pressed to tablets in potassium phosphate buffer (control) and artificial gastric juice and intestinal juice. The 100% value was set to the total active ingredient concentrations shown in the table according to example 10.

### DETAILED DESCRIPTION OF THE INVENTION

1. Definition of Terms Used

Unless stated otherwise, the following definitions of technical terms apply in the context of the present invention:

A “carrier polymer” is understood to mean synthetic polymers or blends thereof, biopolymers or blends thereof, or else blends of at least one synthetic polymer and a biopolymer, the carrier polymer having the ability to enter into noncovalent interactions with the active ingredient(s)/effect substance(s) to be formulated, or to surround particulate active ingredients (in dispersed or crystalline form).

A “noncovalent” interaction is understood to mean all types of bonds known to those skilled in the art which do not involve formation of covalent bonds between active ingredient and carrier polymer. Nonlimiting examples thereof include the following: hydrogen bond formation, complex formation, ionic interaction.

An “active ingredient” or “effect substance” is understood to mean synthetic or natural, low molecular weight substances with hydrophilic, lipophilic or amphiphilic properties, which can find use in agrochemistry, pharmacy, cosmetics or the foods and feeds industry; and likewise biologically active macromolecules which can be embedded into or adsorbed onto an inventive fibrous sheetlike structure, for example peptides (such as oligopeptides having 2 to 10 amino acid residues and polypeptides having more than 10, for example 11 to 100, amino acid residues), and also enzymes and single- or double-strand nucleic acid molecules (such as oligonucleotides having 2 to 50 nucleic acid residues and polynucleotides having more than 50 nucleic acid residues).

“Low molecular weight” means molar masses of less than 5000, especially less than 2000, for example 100 to 1000, grams per mole.

“High molecular weight” means molar masses of more than 5000, especially less than 10 000, for example 10 000 to 1 000 000, grams per mole.

The terms “active ingredient” and “effect substance” are used synonymously.

According to the invention, the term “fibrous sheetlike structure” comprises both individual polymer fibers and the combination of a multitude of such fibers, for example to give fiber nonwovens.

An “active ingredient carrier” is in fibrous form and bears, preferably in adsorbed, noncovalently bonded form on the fiber surface and/or integrated into the fiber material, the active ingredient(s) to be processed in accordance with the invention. The active ingredient may be present in homogeneous or inhomogeneous distribution over the fiber. The active ingredient may additionally be reversibly adsorbed in amorphous, semicrystalline or crystalline form on/in the active ingredient carrier.

A “soluble” active ingredient carrier is partly or fully soluble in an aqueous or organic solvent, preferably an aqueous solvent, for example water or a water-based solvent, within a pH range of pH 2 to 13, for example 4 to 11. Thus, the solubility in water can vary within a wide range—i.e. from good, i.e. rapid and complete or essentially complete solubility to very slow and complete or incomplete solubility.

Suitable polymeric constituents of the inventive active ingredient formulations are in principle all polymers which are soluble in water or/and in organic solvents within a temperature range between 0 and 240° C., a pressure range between 1 and 100 bar, a pH range from 0 to 14 or ionic strengths up to 10 mol/l.

A “degradable” active ingredient carrier is present when the fiber structure is partly or completely destroyed by chemical, biological or physical processes, for example by the action of light or other radiation, solvents, chemical or biochemical oxidation, hydrolysis, proteolysis.

Biochemical processes can be mediated by enzymes or microorganisms, for example by prokaryotes or eukaryotes, for example bacteria, yeasts, fungi.

“Miscibility” of polymers is understood in accordance with the invention to mean that, in the case of a mixture of at least two different polymers, one polymer can function as a solvent for the other. This means that a monophasic system forms between the two different polymers. In the case of immiscible components, two different phases are correspondingly present.

A “composite polymer” is understood in accordance with the invention to mean a homogeneous or inhomogeneous mixture of at least one fiber-forming polymer component with at least one low molecular weight or high molecular weight additive, such as especially a nonpolymerizable additive, for example an active ingredient or effect substance as defined above.

A “processed form” of a fibrous sheetlike structure is understood to mean that the product originally obtained in the production of the fibrous sheetlike structure is processed further; for example that the fibers are compressed or tableted, applied to a further carrier and/or subjected to a comminution to shorten the fiber length.

Unless stated otherwise, molecular weight figures for polymers relate to Mn or Mw values.

2. Preferred Embodiments

The invention firstly relates to active ingredient-containing fibrous sheetlike structures comprising a fibrous, polymeric, soluble and/or degradable active ingredient carrier and a low molecular weight active ingredient which is associated with the carrier and can be released by the fibrous sheetlike structure, or a plurality of active ingredients, for example 2, 3, 4 or 5 active ingredients, from the same or different active ingredient classes or with the same or different mode of action, wherein the carrier is a composite polymer which comprises a mixture of two or more, for example 2, 3, 4 or 5, polymer components, wherein these at least two polymer components differ in at least one property which is selected from

a) solubility in aqueous or nonaqueous solvents,

b) molecular weight (Mn or Mw)

c) glass transition temperature (Tg)/melting point (m.p.); and

d) degradability, such as chemical degradability and especially biodegradability, for example by at least one enzyme or at least one microorganism, oxidatively and/or hydrolytically and/or by radiation.

More particularly, the polymer components differ with regard to solubility and/or degradability.

In addition, the at least two polymer components may differ by

(i) differing loading densities with the active ingredient(s);

(ii) different specific surface area of the fibers (i.e. diameter);

(iii) different physical structure of the fibers, for example in the form of different porosity and/or surface roughness (topography), phase separation.

More particularly, the at least one active ingredient is in amorphous or semicrystalline form.

For example, the active ingredient in the fibrous sheetlike structure may be integrated (embedded) into and/or absorbed onto the carrier.

Especially preferably, the fibrous, active ingredient-containing carrier is obtainable by a spinning process.

More particularly, the fibrous, active ingredient-containing carrier is produced by an electrospinning process with an electrospinnable solution comprising, in each case in dissolved form, the at least one active ingredient and the mixture of at least two polymer components.

The polymer components present in the inventive fibrous sheetlike structure are miscible with one another, or at least two of the polymer components are immiscible with one another.

The polymer components used in accordance with the invention are especially selected from synthetic polymers and natural polymers (biopolymers), such as especially amphiphilic self-assembly proteins, wherein the biopolymers may additionally have been chemically and/or enzymatically modified.

The amphiphilic self-assembly proteins are, for example, microbead-forming proteins, or intrinsically unfolded proteins. For example, the amphiphilic self-assembly protein is a silk protein, such as especially a spider silk protein, preferably a C16, R16 or S16 protein (cf. SEQ ID NO: 2, 4 or 6); or a spinnable protein derived from these proteins having a sequence identity of at least about 50%, for example at least 60, 70, 80, 90, 95, 96, 97, 98 or 99% sequence identity.

The synthetic polymer is either a homo- or copolymer.

The carrier polymer is especially selected from

a) mixtures of at least 2 miscible synthetic homo- or copolymers;

b) mixtures of at least 2 immiscible synthetic homo- or copolymers;

c) mixtures of at least 2 miscible biopolymers;

d) mixtures of at least 2 immiscible biopolymers;

e) mixtures of at least one synthetic homo- or copolymer and at least one biopolymer which are miscible with one another; and

f) mixtures of at least one synthetic homo- or copolymer and at least one biopolymer which are immiscible with one another.

The polymer components each independently have molar masses in the range from about 500 to 10 000 000, for example 1000-1 000 000 or 10 000-500 000 or 20 000-250 000.

The diameter of the active ingredient carrier fibers is about 10 nm to 100 μm, for example 50 nm to 10 μm, or 100 nm to 2 μm.

In addition, in accordance with the invention, the active ingredient loading may be about 0.01 to 80% by weight, for example 1 to 70% by weight or 10 to 50% by weight, based on the solids content of the fibrous sheetlike structure.

More particularly, the inventive fibrous sheetlike structure is selected from polymer fibers and polymer nonwovens.

In addition, the fibers may have additional physical structuring, for example porosity. Moreover, to increase the viscosity or viscoelasticity, and for better spinnability of the solution, at least one further polymer may be present. In addition, at least one low molecular weight additive, for example an organic or inorganic salt to increase the electrical conductivity of the spinnable solution, penetration aids for active ingredients, assistants for increasing bioavailability, etc., may be present.

In the fibrous sheetlike structures, the active ingredient is especially present in the fibers in molecular dispersion (i.e. the active ingredient molecules are present individually in the polymer matrix, i.e. are dissolved therein) or in nanoparticulate dispersion (i.e. the molecules are aggregated to particles (clusters) with dimensions in the range of a few nanometers).

The invention also provides active ingredient-containing formulations comprising a fibrous sheetlike structure as defined above in processed form, optionally in combination with at least one further formulating assistant, which comprises the fibrous sheetlike structure in comminuted or noncomminuted form. For example, the fibrous sheetlike structure may be present in compacted (pressed) form (such as tablets or capsules), in powder form, or applied to a carrier substrate.

Inventive formulations are especially selected from cosmetic (especially skin- and hair-cosmetic) formulations, human and animal pharmaceutical formulations, agrochemical formulations (especially fungicides, herbicides, insecticides and other crop protection formulations), food and animal feed additives (for example food and animal feed supplements).

The invention also provides for the use of an active ingredient-containing fibrous sheetlike structure as defined above for production of an active ingredient-containing formulation as defined above, and more particularly for the use of an active ingredient-containing formulation as defined above for controlled release of an active ingredient present therein.

Finally, the invention provides a process for producing a fibrous sheetlike structure as defined above, wherein:

a) the at least one active ingredient is mixed together with the carrier polymer components in a combined liquid phase and

b) then the embedding of the active ingredient into a polymeric composite fiber is performed by means of spinning processes.

This involves mixing the at least one active ingredient and the polymer components in a solvent phase and spinning this mixture.

However, it is also possible to mix the at least one active ingredient and the polymer components in a mixture of at least two mutually miscible solvents, active ingredients and polymers being soluble at least in one of the solvents, and to spin the mixture thus obtained.

The spinning process may be an electrospinning process or a centrifuge (rotor) spinning process. More particularly, the spinning process is performed at a temperature in the range from about 0 to 90° C.

The present invention also relates to the fibrous sheetlike structure wherein

(i) the diameter of the fibers is 10 nm to 100 μm, preferably 50 nm to 10 μm, more preferably 100 nm to 2 μm,

(ii) the effect substance loading is from 0.01 to 80% by weight, preferably 1 to 60% by weight, more preferably 5 to 50% by weight, based on the total solids of the formulation,

(iii) the effect substance is present in x-ray-amorphous or semicrystalline form (as a fine dispersion) in the fibers together with the polymers and optionally additives.

For particular applications, it could be advantageous if the polymers separate after the removal of the common solvent to form two or more phases.

Spinning operations can be used to produce sheetlike structures (fibers, nonwovens, coatings) from aqueous solutions or organic solvents in which synthetic polymers or biopolymers and effect substances are present in dissolved or dispersed form.

These polymer- and active ingredient-rich phases can be used in the form of coatings (layers on a substrate), removed in the form of mechanically stable active ingredient-containing polymer structures and optionally dried, and also processed to tablets or capsules.

The invention also provides fibrous sheetlike structures as defined above, which are essentially free of low molecular weight active ingredients and/or high molecular weight active ingredients.

The invention finally relates to the use of such an active ingredient-free fibrous sheetlike structure for production of an active ingredient-containing formulation, in which case the formulation is especially selected from cosmetic formulations, human and animal pharmaceutical formulations, agrochemical formulations, food and animal feed additives.

For this purpose, for example, the active ingredient-free fibrous sheetlike structure can be produced essentially as described herein by spinning suitable polymers and, in a next step, one or more active ingredients can be associated therewith, for example adsorbed, i.e. bound noncovalently.

3. Further Configurations of the Invention

(i) Formulation of Active Ingredients

The inventive formulations of active ingredients can be produced by known methods using synthetic polymers and/or biopolymers in various ways. The active ingredients can be packaged or encapsulated in fibrous sheetlike structures, for example, by spinning processes.

The fibers and sheetlike structures composed of polymer-active ingredient combinations can be produced proceeding from a solution or a finely divided dispersion or a gel by all spinning processes known to those skilled in the art. Particularly suitable spinning processes are those from solution or a finely divided dispersion, more preferably including centrifuge spinning (rotor spinning) and electrospinning (electrostatic spinning).

In the case of spinning of formulations to fibers, suitable fiber diameters in principle are from 10 nm to 100 μm, preferably diameters from 50 nm to 10 μm, more preferably from 100 nm to 2 μm.

In the case of electrospinning (electrostatic spinning), the solution or finely divided dispersion to be formulated is introduced into an electrical field of strength between 0.01 and 10 kV/cm, more preferably between 1 and 6 kV/cm and most preferably between 2 and 4 kV/cm. As soon as the electrical forces exceed the surface tension of the formulation, mass is transferred in the form of a jet to the opposite electrode. The solvent evaporates in the space between the electrodes, and the solids in the formulation are then present in the form of fibers on the counterelectrode. The spinning electrode may be die- or syringe-based or have roller geometry. The spinning can be effected in either vertical direction (from the bottom upward and from the top downward), and in horizontal direction.

A further process suitable in accordance with the invention is centrifuge spinning (rotor spinning). In this process, the starting material is introduced as a solution or finely divided dispersion into a field with gravitational forces. For this purpose, the fiber raw material is introduced into a vessel and the vessel is set to rotate, in the course of which the fluidized fiber raw material is discharged from the vessel in the form of fibers by centripetal or centrifugal forces. The fibers can subsequently be transported away by gas flow and combined to form sheetlike structures.

The active ingredients can be formulated in accordance with the invention by inclusion into the fibrous sheetlike structures produced by the process according to the invention. This process usually comprises two steps. In the first step, a spinning solution is prepared from active ingredient(s) and carrier polymer(s) by mixing the components in a common phase. For this purpose, active ingredient and polymers can be brought into solution directly by means of a solvent or a solvent mixture. Alternatively, active ingredient and polymers can first be dissolved in different solvents and the solutions can then be mixed with one another, so as again to give rise to a common phase. The common phase may also be a molecularly disperse phase or a colloidally disperse phase.

The dissolution of active ingredient and polymer in different solvents and the subsequent mixing of the two solutions are especially advantageous when active ingredient and polymer cannot be dissolved in a common solvent or solvent mixture. In this way, it is also possible to produce colloidally disperse solutions of hydrophobic active ingredients, by diluting the active ingredient dissolved in a suitable solvent in another solvent in which this active ingredient is insoluble.

Suitable solvents should in principle not hinder the formation of fibrous sheetlike structures and not irreversibly inactivate the active ingredient.

Useful solvents include firstly water, and also mixtures of water and water-miscible organic solvents. Examples of suitable water-miscible solvents are, without any restriction, alcohols such as methanol, ethanol and isopropanol, fluorinated alcohols such as hexafluoroisopropanol and trifluoroethanol, alkanones such as acetone; or else sulfoxides, for example dimethyl sulfoxide; or formamides such as dimethylformamide; or other organic solvents, for example tetrahydrofuran and acetonitrile or N-methyl-2-pyrrolidone or formate. In general, it is possible to work with any solvents and solvent mixtures in which the carrier polymers can be dissolved. Further examples of suitable solvents are ionic liquids, for example 1-ethyl-3-methylimidazolium (EMIM) acetate, aqueous solutions of chaotropic salts, for example urea, guanidinium hydrochloride and guanidinium thiocyanate, or organic acids, for example formic acid, acetic acid, etc.

In a further embodiment, it is possible to use solvents or solvent mixtures which are immiscible with water. The term “water-immiscible organic solvent” describes organic solvents which have a solubility in water of less than 50%, preferably less than 25%, more preferably less, than 10%, even more preferably less than 10%, in an exceptionally preferred embodiment less than 5%.

The following solvents are mentioned by way of example, but without any restriction: cyclohexane, cyclopentane, pentane, hexane, heptane, 2-methylpentane, 3-methylpentane, 2-methylhexane, 3-methylhexane, 2-methylbutane, 2,3-dimethylbutane, methylcyclopentane, methylcyclohexane, 2,3-dimethylpentane, 2,4-dimethylpentane, benzene, 1-pentene, 2-pentene, 1-hexene, 1-heptene, cyclohexene, 1-butanol, ethyl vinyl ether, propyl ether, isopropyl ether, butyl vinyl ether, butyl ethyl ether, 1,2-epoxybutane, furan, tetrahydropyran, 1-butanal, 2-methylpropanal, 2-pentanone, 3-pentanone, cyclohexanone, fluorobenzene, hexafluorobenzene, ethyl formate, propyl formate, isopropyl formate, ethyl acetate, vinyl acetate, isopropyl acetate, ethyl propionate, methyl acrylate, ethyl acrylate, methyl methacrylate, chloroethane, 1-chloropropane, 2-chloropropane, 1-chlorobutane, 2-chlorobutane, 1-chloro-2-methylpropane, 2-chloro-2-methylpropane, 1-chloro-3-methylbutane, 3-chloropropene, dichloromethane, trichloromethane, tetrachloromethane, 1-dichloroethane, 1,2-dichloroethane, 1,2-dichloropropane, 1,1,1-trichloroethane, 1,2-dichloroethylene, 1,2-dichloroethylene, trichloroethylene, bromomethane, 1-bromopropane, 2-bromopropane, 1-bromobutane, 2-bromobutane, 2-bromo-2-methylpropane, bromomethylene, iodomethane, iodoethane, 2-iodopropane, trichlorofluoromethane, dichlorofluoromethane, dibromofluoromethane, bromochloromethane, bromochlorofluoromethane, 1,1,2-trichloro-1,2,2-trifluoroethane, 1,1,2,2-tetrachlorodifluoroethane, 1,2-dibromotetrafluoroethane, 1,2-dibromo-1-1-diflouroethane, 1,1-dichloro-2,2-difluoroethylene, propionitrile, acrylonitrile, methacrylonitrile, triethylamine, carbon disulfide, 1-butanethiol, methyl sulfide, ethyl sulfide and tetramethylsilane.

ii) Fibrous Sheetlike Structures and Polymer Components Thereof

The inventive fibers in fibrous sheetlike structures may consist of one, two, three or more phases.

In a further embodiment of the present invention, the fiber of the inventive fibrous sheetlike structure consists of at least three phases, in which case one phase consists of amorphous or semicrystalline or crystalline particles of the active ingredient, the other phase constitutes a molecularly disperse distribution of the active ingredient in a polymer matrix, and the third phase constitutes an active ingredient-free polymer phase.

In a further embodiment of the present invention, the fiber of the inventive fibrous structure consists of at least two phases, in which case one phase consists of amorphous or semicrystalline or crystalline particles of the active ingredient, the other phase constitutes a molecularly disperse distribution of the active ingredient in a polymer matrix.

In a further embodiment of the present invention, the fiber of the inventive fibrous sheetlike structures consists of at least two phases, in which case one phase consists of amorphous or semicrystalline or crystalline active ingredient, and the other phase constitutes an active ingredient-free polymer matrix.

In a further preferred embodiment of the present invention, the fiber of the inventive fibrous sheetlike structure consists of a molecularly disperse distribution of the active ingredient in a polymer matrix.

In the case of use of immiscible polymers A and B, further phases can be formed, which consist, for example, of active ingredient and polymer A with small amounts of polymer B, or of active ingredient and polymer B with small amounts of polymer A.

Suitable polymeric constituents of the inventive active ingredient formulations are in principle all natural and synthetic polymers which are soluble in water or/and in organic solvents within a temperature range between 0 and 240° C., a pressure range between 1 and 100 bar, a pH range from 0 to 14 or ionic strengths up to 10 mol/l. it is possible to use one or more polymers. The molar masses of the polymers used are in the range of 500-10 000 000 g/mol, preferably in the range of 1000-1 000 000 g/mol. Useful polymers in principle are all polymers suitable for the application sectors of pharmacology, crop protection, cosmetics, food and animal feed production.

The polymers with high molecular weight (from 500 000) are advantageous when a sparingly soluble effect substance is to be formulated. These polymers require a very low concentration in the formulation to obtain fibrous sheetlike structures therefrom. The effect substance concentration in the formulation will also be correspondingly low.

If the intention is to obtain formulations of an amorphous active ingredient with improved long-term stability, the polymers should either have a strong noncovalent interaction with active ingredient or have a glass transition temperature (Tg) preferably above the spinning temperature. In this case, the active ingredient remains dissolved in molecular dispersion or fine dispersion in the polymer after the removal of the solvent, since in the first case the interaction with the carrier and in the second case the lack of mobility of the polymer chains below the glass transition temperature hinder the movement of the active ingredient molecules. It is optionally also possible for at least one additive to be present, which hinders the agglomeration of the active ingredient.

Suitable synthetic polymers are, for example, selected from the group consisting of homo- and copolymers of aromatic vinyl compounds, homo- and copolymers of alkyl acrylates, homo- and copolymers of alkyl methacrylates, homo- and copolymers of α-olefins, homo- and copolymers of aliphatic dienes, homo- and copolymers of vinyl halides, homo- and copolymers of vinyl acetates, homo- and copolymers of acrylonitriles, homo- and copolymers of urethanes, homo- and copolymers of vinylamides and copolymers formed from two or more of the monomer units forming the aforementioned polymers.

Useful carrier polymers include more particularly polymers based on the following monomers:

acrylamide, adipic acid, allyl methacrylate, alpha-methylstyrene, butadiene, butanediol, butanediol dimethacrylate, butanediol divinyl ether, butanediol dimethacrylate, butanediol monoacrylate, butanediol monomethacrylate, butanediol monovinyl ether, butyl acrylate, butyl methacrylate, cyclohexyl vinyl ether, diethylene glycol divinyl ether, diethylene glycol monovinyl ether, ethyl acrylate, ethyldiglycol acrylate, ethylene, ethylene glycol butyl vinyl ether, ethylene glycol dimethacrylate, ethylene glycol divinyl ether, ethylhexyl acrylate, ethylhexyl methacrylate, ethyl methacrylate, ethyl vinyl ether, glycidyl methacrylate, hexanediol divinyl ether, hexanediol monovinyl ether, isobutene, isobutyl acrylate, isobutyl methacrylate, isoprene, isopropylacrylamide, methyl acrylate, methylenebisacrylamide, methyl methacrylate, methyl vinyl ether, n-butyl vinyl ether, N-methyl-N-vinylacetamide, N-vinylcaprolactam, N-vinylimidazole, N-vinylpiperidone, N-vinylpyrrolidone, octadecyl vinyl ether, phenoxyethyl acrylate, polytetrahydrofuran 2 divinyl ether, propylene, styrene, terephthalic acid, tert-butylacrylamide, tert-butyl acrylate, tert-butyl methacrylate, tetraethylene glycol divinyl ether, triethylene glycol dimethyl acrylate, triethylene glycol divinyl ether, triethylene glycol divinyl methyl ether, trimethylolpropane trimethacrylates, trimethylolpropane trivinyl ether, vinyl 2-ethylhexyl ether, vinyl 4-tert-butylbenzoate, vinyl acetate, vinyl chloride, vinyl dodecyl ether, vinylidene chloride, vinyl isobutyl ether, vinyl isopropyl ether, vinyl propyl ether and vinyl tert-butyl ether.

The term “polymers” comprises both homopolymers and copolymers. Useful copolymers are not only random but also alternating systems, block copolymers or graft copolymers. The term “copolymers” comprises polymers formed from two or more different monomers, or else where the incorporation of at least one monomer into the polymer chain can be realized in various ways, as is the case with stereoblock copolymers for example.

It is also possible to use blends of homo- and copolymers. The homo- and copolymers may or may not be miscible with each other.

The following polymers should be mentioned with preference:

polyvinyl ethers, for example polybenzyloxyethylene, polyvinyl acetals, polyvinyl esters, for example polyvinyl acetate, polyoxytetramethylene, polyamides, polycarbonates, polyesters, polysiloxanes, polyurethanes, polyacrylamides, for example poly(N-isopropylacrylamide), polymethacrylamides, polyhydroxybutyrates, polyvinyl alcohols, acetylated polyvinyl alcohols, polyvinylformamide, polyvinylamines, polycarboxylic acids (polyacrylic acid, polymethacrylic acid), polyacrylamide, polyitaconic acid, poly(2-hydroxyethyl acrylate), poly(N-isopropylacrylamide), polysulfonic acid (poly(2-acrylamido-2-methyl-1-propanesulfonic acid) or PAMPS), polymethacrylamide, polyalkylene oxides, e.g., polyethylene oxides; poly-N-vinylpyrrolidone; maleic acids, poly(ethyleneimine), polystyrenesulfonic acid, polyacrylates, e.g. polyphenoxyethyl acrylate, polymethyl acrylate, polyethyl acrylate, polydodecyl acrylate, poly(ibornyl acrylate), poly(n-butyl acrylate), poly(t-butyl acrylate), polycyclohexyl acrylate, poly(2-ethylhexyl acrylate), polyhydroxypropyl acrylate, polymethacrylates, e.g. polymethyl methacrylate, poly(n-amyl methacrylate), poly(n-butyl methacrylate), polyethyl methacrylate, poly(hydroxypropyl methacrylate), polycyclohexyl methacrylate, poly(2-ethylhexyl methacrylate), polylauryl methacrylate, poly(t-butyl methacrylate), polybenzyl methacrylate, poly(ibornyl methacrylate), polyglycidyl methacrylate and polystearyl methacrylate, polystyrene, and also copolymers based on styrene, for example with maleic anhydride, styrene-butadiene copolymers, methyl methacrylate-styrene copolymers, N-vinylpyrrolidone copolymers, polycaprolactones, polycaprolactams, poly(N-vinylcaprolactam).

Very particular preference is given to poly-N-vinylpyrrolidone, polymethyl methacrylate, acrylate-styrene copolymers, polyvinyl alcohol, polyvinyl acetate, polyamide and polyester.

Additionally suitable are natural polymers or biopolymers:

Nonlimiting examples include: cellulose, cellulose ethers, for example methyl cellulose (degree of substitution 3-40%), ethyl cellulose, butyl cellulose, hydroxymethyl celluloses; hydroxyethyl celluloses; hydroxypropyl celluloses, isopropyl cellulose, cellulose esters, for example cellulose acetate, starches, modified starches, for example methyl ether starch, gum arabic, chitin, schellack, gelatin, chitosan, pectin, casein, alginate, and copolymers and block copolymers formed from the monomers of the abovementioned compounds; and nucleic acid molecules.

More particularly, biopolymers used in accordance with the invention are biodegradable.

Further suitable biodegradable biopolymers are amphiphilic, self-assembly proteins. Amphiphilic, self-assembly proteins consist of polypeptides formed from amino acids, especially from the 20 naturally occurring amino acids. The amino acids may also be modified, for example acetylated, glycosylated, farnesylated.

Suitable amphiphilic, self-assembly proteins are especially those proteins which can form protein microbeads and which are described in WO-A-20077082936, which is explicitly incorporated here by reference.

Further suitable proteins for the formulation of active ingredients by means of spinning processes are silk proteins. We understand these hereinafter to mean those proteins which comprise highly repetitive amino acid sequences and are stored in a liquid form in the animal, the secretion of which gives rise to fibers as a result of shearing or spinning (Craig, C. L. (1997) Evolution of arthropod silks. Annu. Rev. Entomol. 42: 231-67).

Particularly suitable proteins for the formulation of active ingredients by means of spinning processes are spider silk proteins which have been isolated in their original form from spiders.

Very particularly suitable proteins are silk proteins which have been isolated from the major ampullate gland of spiders.

Preferred silk proteins are ADF3 and ADF4 from the major ampullate gland of Araneus diadematus (Guerette et al., Science 272, 5258:112-5 (1996)).

Equally suitable proteins for the formulation of active ingredients by means of spinning processes are natural or synthetic proteins which derive from natural silk proteins and which have been produced heterologously in prokaryotic or eukaryotic expression systems using genetic engineering methods. Nonlimiting examples of prokaryotic expression organisms are Escherichia coli, Bacillus subtilis, Bacillus megaterium, Corynebacterium glutamicum inter alia. Nonlimiting examples of eukaryotic expression organisms are yeasts, such as Saccharomyces cerevisiae, Pichia pastoris inter alia, filamentous fungi such as Aspergillus niger, Aspergillus oryzae, Aspergillus nidulans, Trichoderma reesei, Acremonium chrysogenum inter alia, mammalian cells such as hela cells, COS cells, CHO cells inter alia, insect cells such as Sf9 cells, MEL cells inter alia.

Particularly preferred for the formulation of active ingredients by means of spinning processes are synthetic proteins based on repeat units from natural silk proteins. In addition to the synthetic repetitive silk protein sequences, they may additionally comprise one or more natural nonrepetitive silk protein sequences (Winkler and Kaplan, J Biotechnol 74:85-93 (2000)).

Among the synthetic silk proteins, for the formulation of active ingredients by means of spinning processes, preference is given to synthetic spider silk proteins based on repeat units from natural spider silk proteins. In addition to the synthetic repetitive spider silk protein sequences, they may additionally comprise one or more natural nonrepetitive spider silk protein sequences.

Among the synthetic spider silk proteins, mention should preferably be made of C16 protein (Huemmerich et al. Biochemistry, 43(42):13604-13612 (2004)). This protein has the polypeptide sequence shown in SEQ ID NO: 2.

In addition to the polypeptide sequence shown in SEQ ID NO: 2, preference is also given particularly to functional equivalents, functional derivatives and salts of this sequence.

Additionally preferred for the formulation of active ingredients by means of spinning processes are synthetic proteins based on repeat units from natural silk proteins combined with sequences from insect structure proteins such as resilin (Elvin et al., 2005, Nature 437: 999-1002).

Among these combination proteins composed of silk proteins and resilins, particular preference is given to the R16 and S16 proteins. These proteins have the polypeptide sequences shown in SEQ ID NO: 4 and SEQ ID NO: 6 respectively.

In addition to the polypeptide sequences shown in SEQ ID NO: 4 and SEQ ID NO: 6, preference is also given particularly to functional equivalents, functional derivatives and salts of these sequences.

“Functional equivalents” are understood in accordance with the invention especially to include mutants which have a different amino acid than that specified in at least one sequence position of the abovementioned amino acid sequences but nevertheless have the property of packaging effect substances. “Functional equivalents” thus comprises the mutants obtainable by one or more amino acid additions, substitutions, deletions and/or inversions, where the changes mentioned may occur in any sequence position provided that they lead to a mutant with the inventive profile of properties. Functional equivalence exists especially also when the reactivity patterns correspond in qualitative terms between mutant and unchanged polypeptide.

“Functional equivalents” in the above sense are also “precursors” of the polypeptides described, and “functional derivatives” and “salts” of the polypeptides.

“Precursors” are natural or synthetic precursors of the polypeptides with or without the desired biological activity.

Examples of suitable amino acid substitutions can be taken from the following table:

| Original residue | Examples of substitution |
|---|---|
| Ala | Ser |
| Arg | Lys |
| Asn | Gln; His |
| Asp | Glu |
| Cys | Ser |
| Gln | Asn |
| Glu | Asp |
| Gly | Pro |
| His | Asn; Gln |
| Ile | Leu; Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

The expression “salts” is understood to mean both salts of carboxyl groups and acid addition salts of amino groups of the inventive protein molecules. Salts of carboxyl groups can be prepared in a manner known per se and comprise inorganic salts, for example sodium, calcium, ammonium, iron and zinc salts, and salts with organic bases, for example amines, such as triethanolamine, arginine, lysine, piperidine and the like. Acid addition salts, for example salts with mineral acids, such as hydrochloric acid or sulfuric acid, and salts with organic acids, such as acetic acid and oxalic acid, likewise form part of the subject matter of the invention.

“Functional derivatives” of inventive polypeptides can likewise be prepared on functional amino acid side groups or on the N- or C-terminal end thereof with the aid of known techniques. Such derivatives comprise, for example, aliphatic esters of carboxylic acid groups, amides of carboxylic acid groups, obtainable by reaction with ammonia or with a primary or secondary amine; N-acyl derivatives of free amino groups, prepared by reaction with acyl groups; or O-acyl derivatives of free hydroxyl groups, prepared by reaction with acyl groups.

“Functional equivalents” also encompassed in accordance with the invention are homologs to the proteins/polypeptides disclosed specifically herein. These have at least 60%, for example 70, 80 or 85%, for example 90, 91, 92, 93, 94, 95, 96, 97, 98 or 99%, identity to one of the amino acid sequences disclosed specifically.

“Identity” between two sequences is understood especially to mean the identity of the radicals over the overall sequence length in each case, especially the identity which is calculated by comparison with the aid of the Vector NTI Suite 7.1 (Vector NTI Advance 10.3.0, Invitrogen Corp.) (or software from Informax (USA) using the clustal method (Higgins D G, Sharp P M. Fast and sensitive multiple sequence alignments on a microcomputer. Comput Appl. Biosci. 1989 Apr;5(2):151-1)) with the following parameter settings:

Multiple alignment parameter:

| | |
|---|---|
| Gap opening penalty | 10 |
| Gap extension penalty | 0.05 |
| Gap separation penalty range | 8 |
| Gap separation penalty | off |
| % identity for alignment delay | 40 |
| Residue specific gaps | off |
| Hydrophilic residue gap | off |
| Transition weighing | 0 |

Pairwise alignment parameter:

| FAST algorithm | off |
|---|---|
| K-tuple size | 1 |
| Gap penalty | 3 |
| Window size | 5 |
| Number of best diagonals | 5 |

Also of particular interest are synthetic biodegradable polymers.

The term “biodegradable polymers” shall comprise all polymers that meet the biodegradability definition given in DIN V 54900, more particularly compostable polyesters.

The general meaning of biodegradability is that the polymers, such as polyesters for example, decompose within an appropriate and verifiable interval. Degradation may be effected hydrolytically and/or oxidatively and predominantly through the action of microorganisms, such as bacteria, yeasts, fungi and algae. Biodegradability can be quantified, for example, by polyesters being mixed with compost and stored for a certain time. According to ASTM D 5338, ASTM D 6400 and DIN V 54900 CO₂-free air is, for example, flowed through ripened compost during composting and the ripened compost subjected to a defined temperature program. Biodegradability here is defined via the ratio of the net CO₂ released by the sample (after deduction of the CO₂ released by the compost without sample) to the maximum amount of CO₂ releasable by the sample (calculated from the carbon content of the sample). Biodegradable polyesters typically show clear signs of degradation, such as fungal growth, cracking and holing, after just a few days of composting. Examples of biodegradable polymers are biodegradable polyesters, for example polylactide, polycaprolactone, polyalkylene adipate terephthalates, polyhydroxyalkanoates (polyhydroxybutyrate) and polylactide glycoside. Particular preference is given to biodegradable polyalkylene adipate terephthalates, preferably polybutylene adipate terephthalates. Suitable polyalkylene adipate terephthalates are described for example in DE 4 440 858 (and are commercially available, e.g., Ecoflex from BASF).

The polymer structures can be produced as active ingredient-comprising fibrous sheetlike structures (e.g. polymer fibers, polymer nonwovens) and laid during the spinning operation onto substrates, for example microfiber nonwovens. Subsequently, these can be pressed to tablets or capsules.

It is additionally possible to add further substances to the spinning solution in order, for example, to influence crystallization of the active ingredient in the fibers at a later stage (for example to inhibit it) or to achieve particular use properties, such as bioavailability.

Preferred additives are, for example, ionic (cationic or anionic) and nonionic surfactants. Suitable amounts of the additives in the spinning solution are 0,01% by weight to 5% by weight.

In addition, it is possible to add to the spinning solution or to the sheetlike polymer structures produced therefrom substances which enable disintegration of the tablets or capsules and hence improved dispersion of the sheetlike polymer structures pressed to the tablets or capsules.

According to the invention, it has been observed more particularly that suitable combination of polymer components for the formulation of the inventive active ingredient-containing fibrous sheetlike structure can influence the active ingredient release properties thereof in a controlled manner. More particularly, this is done by combining at least two polymer components which differ in at least one of the following properties:

a) solubility in aqueous or nonaqueous solvents,

b) molecular weight

c) glass transition temperature and/or melting point

d) degradability (especially biodegradability or chemical degradability, it being possible to induce biodegradability especially by means of at least one enzyme or a microorganism, and chemical degradability being possible, for example, by a hydrolytic or oxidative route. In addition, degradability can also be induced physically, such as especially by the action of light.)

In this way, the present invention can be utilized to adjust the active ingredient release to the particular requirement of the user. The release profile to be provided in each case can be determined empirically on the basis of systematic considerations or by a few preliminary tests. As explained in detail in the working examples present, it is possible in accordance with the invention, by combining different polymer components, to obtain new release profiles for active ingredients which differ significantly from the release profiles observed for the individual polymer components. For example, it is possible to observe distinctly earlier onset of active ingredient release by the polymer combination compared to release by the individual polymer components, or a higher or lower release rate in the release profile for inventive polymer combinations compared to the individual components over the entire or over part of the observation period.

Nonlimiting examples of particularly suitable polymer combinations are, in addition to the combinations illustrated in the examples, as follows:
Polyester/polyacrylate (immiscible)
Polyester/polystyrene or styrene copolymer (acrylate or butadiene) (immiscible)
Polyamide/spider silk protein (miscible)
Styrene or styrene copolymer/spider silk protein
PVP/spider silk protein (miscible)
PVA/spider silk protein
PEO/spider silk protein
Polyamide/PVP (miscible)
Polyamide/polyacrylic acid (miscible)
Polylactic acid/PVP (possibly immiscible)
Polylactic acid/polyacrylate
Polyester/polyacrylate/PVP (immiscible)
Polyester/polylactic acid/PVP (immiscible or miscible)
Polyester/starch (miscible)
PVP/starch
Cellulose or derivatives/polyacrylate
Cellulose acetate/polyethylene-vinyl acetate (miscible)
Polyvinyl alcohol/polyvinyl acetate (miscible)
Collagen/PVA
Collagen/PEO
Chitosan/PVA (PEO)
Polyurethane/PVP (miscible)
Polyurethane/polyester (possibly immiscible)
Polyurethane/spider silk protein (miscible)
Polycarbonate/polycaprolactone (miscible)
Polycarbonate/polyester (miscible)
Polyacrylate/polyvinyl chloride (miscible)

(iii) Active Ingredients

The terms “active ingredients” and “effect substances” are used synonymously hereinafter. These are both water-soluble and sparingly water-soluble effect substances. The terms “sparingly water-soluble” and “hydrophobic” active ingredients or effect substances are used synonymously. Sparingly water-soluble active ingredients refer hereinafter to those compounds whose water solubility at 20° C. is <1% by weight, preferably <0.5% by weight, more preferably <0.25% by weight, most preferably <0.1% by weight. Water-soluble active ingredients refer hereinafter to those compounds whose water solubility at 20° C. is >1% by weight, preferably >10% by weight, more preferably >40% by weight, most preferably >70% by weight.

Suitable effect substances are dyes, especially those specified in the following table:

Particularly advantageous dyes are the oil-soluble or oil-dispersible compounds specified in the following list. The color index numbers (CIN) are taken from the Rowe Colour Index, 3rd edition, Society of Dyers and Colourists, Bradford, England, 1971.

| Chemical or other name | CIN | Color |
|---|---|---|
| Pigment Yellow 1 | 11680 | yellow |
| Pigment Yellow 3 | 11710 | yellow |
| Pigment Orange 1 | 11725 | orange |
| 2,4-Dihydroxyazobenzene | 11920 | orange |
| Solvent Red 3 | 12010 | red |
| 1-(2′-Chloro-4′-nitro-1′-phenylazo)-2-hydroxynaphthalene | 12085 | red |
| Pigment Red 3 | 12120 | red |
| Ceres Red; Sudan Red; Fat Red G | 12150 | red |
| Pigment Red 112 | 12370 | red |
| Pigment Red 7 | 12420 | red |
| Pigment Brown 1 | 12480 | brown |
| N-(5-Chloro-2,4-dimethoxyphenyl)-4-[[5-[(diethylamino)sulfonyl]- | 12490 | red |
| 2-methoxyphenyl]azo]-3-hydroxy-2-naphthalenecarboxamide | | |
| Pigment Yellow 16 | 20040 | yellow |
| Pigment Yellow 13 | 21100 | yellow |
| Pigment Yellow 83 | 21108 | yellow |
| Solvent Yellow | 21230 | yellow |
| Food Yellow | 40800 | orange |
| trans-β-Apo-8′-carotinaldehyde (C30) | 40820 | orange |
| trans-Apo-8′-carotinic acid (C30) ethyl ester | 40825 | orange |
| Canthaxanthin | 40850 | orange |
| Solvent Dye | 45396 | orange |
| Quinophthalone | 47000 | yellow |
| Pigment Violet 23 | 51319 | violet |
| 1,2-Dihydroxyanthraquinone, calcium-aluminum complex | 58000 | red |
| 1-Hydroxy-4-N-phenylaminoanthraquinone | 60724 | violet |
| 1-Hydroxy-4-(4′-methylphenylamino)anthraquinone | 60725 | violet |
| 1,4-Di(4′-methyl-phenylamino)anthraquinone | 61565 | green |
| N,N′-Dihydro-1,2,1′,2′-anthraquinonazine | 69800 | blue |
| Vat Blue 6; Pigment Blue 64 | 69825 | blue |
| Vat Orange 7 | 71105 | orange |
| Indigo | 73000 | blue |
| 4,4′-Dimethyl-6,6′-dichlorothioindigo | 73360 | red |
| 5,5′-Dichloro-7,7′-dimethylthioindigo | 73385 | violet |
| Quinacridone Violet 19 | 73900 | violet |
| Pigment Red 122 | 73915 | red |
| Pigment Blue 16 | 74100 | blue |
| Phthalocyanine | 74160 | blue |
| Direct Blue 86 | 74180 | blue |
| Chlorinated phthalocyanine | 74260 | green |
| Bixin, Nor-Bixin | 75120 | orange |
| Lycopene | 75125 | yellow |
| trans-alpha-, -beta- or -gamma-carotene | 75130 | orange |
| Keto and/or hydroxyl derivatives of carotene | 75135 | yellow |
| 1,7-Bis(4-hydroxy-3-methoxyphenyl)1,6-heptadiene-3,5-dione | 75300 | yellow |

Further preferred effect substances are fatty acids, especially saturated fatty acids which bear an alkyl branch, more preferably branched eicosanoic acids such as 18-methyleicosanoic acid.

Further preferred effect substances are carotenoids. Carotenoids are understood in accordance with the invention to mean the following compounds, and the esterified or glycosylated derivatives thereof: β-carotene, lycopene, lutein, astaxanthin, zeaxanthin, cryptoxanthin, citranaxanthin, canthaxanthin, bixin, β-apo-4-carotinal, β-apo-8-carotinal, β-apo-8-carotinic ester, neurosporene, echinenone, adonirubin, violaxanthin, torulene, torularhodin, individually or as a mixture. Carotenoids used with preference are β-carotene, lycopene, lutein, astaxanthin, zeaxanthin, citranaxanthin and canthaxanthin.

Further preferred effect substances are vitamins, especially retinoids and esters thereof.

In the context of the present invention, retinoids mean vitamin A alcohol (retinol) and derivatives thereof, such as vitamin A aldehyde (retinal), vitamin A acid (retinoic acid) and Vitamin A esters (e.g. retinyl acetate, retinyl propionate and retinyl palmitate). The term “retinoic acid” comprises not only all-trans retinoic acid but also 13-cis retinoic acid. The terms “retinol” and “retinal” preferably comprise the all-trans compounds. A preferred retinoid used for the inventive formulations is all-trans-retinol, referred to hereinafter as retinol.

Further preferred effect substances are vitamins, provitamins and vitamin precursors from groups A, B, C, E and F, especially 3,4-didehydroretinol, β-carotene (provitamin of vitamin A), palmitic esters of ascorbic acid, tocopherols, especially α-tocopherol and esters thereof, for example the acetate, the nicotinate, the phosphate and the succinate; and also vitamin F, which is understood to mean essential fatty acids, particularly linolic acid, linolenic acid and arachidonic acid.

Further preferred effect substances are lipophilic, oil-soluble antioxidants from the group of vitamin E, i.e. tocopherol and derivatives thereof, gallic esters, flavonoids and carotenoids, and also butylhydroxytoluene/anisol.

A further preferred effect substance is lipoic acid and suitable derivatives (salts, esters, sugars, nucleotides, nucleosides, peptides and lipids).

Further preferred effect substances are UV light protection filters. This is understood to mean organic substances which are capable of absorbing ultraviolet rays and of releasing the energy adsorbed again in the form of longer-wave radiation, for example heat.

The oil-soluble UV-B filters used may, for example, be the following substances:

3-benzylidenecamphor and derivatives thereof, e.g. 3-(4-methylbenzylidene)camphor; 4-aminobenzoic acid derivatives, preferably 2-ethylhexyl 4-(dimethylamino)benzoate, 2-octyl 4-(dimethylamino)benzoate and amyl 4-(dimethylamino)benzoate; esters of cinnamic acid, preferably 2-ethylhexyl 4-methoxycinnamate, propyl 4-methoxycinnamate, isoamyl 4-methoxycinnamate, isopentyl 4-methoxycinnamate, 2-ethylhexyl 2-cyano-3-phenylcinnamate (octocrylene);

esters of salicylic acid, preferably 2-ethylhexyl salicylate, 4-isopropyl benzyl salicylate, homomenthyl salicylate; derivatives of benzophenone, preferably 2-hydroxy-4-methoxybenzophenone, 2-hydroxy-4-methoxy-4′-methylbenzophenone, 2,2′-dihydroxy-4-methoxybenzophenone; esters of benzalmalonic acid, preferably di-2-ethylhexyl 4-methoxybenzmalonate; triazine derivatives, for example 2,4,6-trianilino-(p-carbo-2′-ethyl-1″-hexyloxy)-1,3,5-triazine (octyltriazone) and Dioctyl Butamido Triazone (Uvasorb® HEB):

propane-1,3-diones, for example 1-(4-tert-butylphenyl)-3-(4′-methoxyphenyl)propane-1,3-dione.
Particular preference is given to the use of esters of cinnamic acid, preferably 2-ethylhexyl 4-methoxycinnamate, isopentyl 4-methoxycinnamate, 2-ethylhexyl 2-cyano-3-phenylcinnamate (octocrylene).

Additionally preferred is the use of derivatives of benzophenone, especially 2-hydroxy-4-methoxybenzophenone, 2-hydroxy-4-methoxy-4′-methylbenzophenone, 2,2′-dihydroxy-4-methoxybenzophenone, and the use of propane-1,3-diones, for example 1-(4-tert-butylphenyl)-3-(4′-methoxyphenyl)propane-1,3-dione.

Useful typical UV-A filters include:

derivatives of benzoyl methane, for example 1-(4′-tert-butylphenyI)-3-(4′-methoxy-phenyl)propan-1,3-dione, 4-tert-butyl-4′-methoxydibenzoylmethane or 1-phenyl-3-(4′-isopropylphenyl)propan-1,3-dione;

amino-hydroxyl-substituted derivatives of benzophenones, for example N,N-diethylamino hydroxybenzoyl n-hexyl benzoate.

The UV-A and UV-B filters may of course also be used in mixtures.

Suitable UV filter substances are specified in the following table:

| | | CAS No. |
|---|---|---|
| No. | Substance | (=acid) |
| 1 | 4-aminobenzoic acid | 150-13-0 |
| 2 | 3-(4′-trimethylammonium)benzylidenebornan-2-one | 52793-97-2 |
| | methylsulfate | |
| 3 | 3,3,5-trimethylcyclohexyl salicylate | 118-56-9 |
| | (homosalate) | |
| 4 | 2-hydroxy-4-methoxybenzophenone | 131-57-7 |
| | (oxybenzone) | |
| 5 | 2-phenylbenzimidazole-5-sulfonic acid and the potassium, | 27503-81-7 |
| | sodium and triethanolamine salts thereof | |
| 6 | 3,3′-(1,4-phenylenedimethine)bis(7,7-dimethyl- | 90457-82-2 |
| | 2-oxobicyclo[2.2.1]heptane-1-methanesulfonic acid) and salts | |
| | thereof | |
| 7 | polyethoxyethyl 4-bis(polyethoxy)aminobenzoate | 113010-52-9 |
| 8 | 2-ethylhexyl 4-dimethylaminobenzoate | 21245-02-3 |
| 9 | 2-ethylhexyl salicylate | 118-60-5 |
| 10 | 2-isoamyl 4-methoxycinnamate | 71617-10-2 |
| 11 | 2-ethylhexyl 4-methoxycinnamate | 5466-77-3 |
| 12 | 2-hydroxy-4-methoxybenzophenone-5-sulfonic acid | 4065-45-6 |
| | (sulisobenzone) and the sodium salt | |
| 13 | 3-(4′-sulfobenzylidene)bornan-2-one and salts | 58030-58-6 |
| 14 | 3-benzylidenebornan-2-one | 16087-24-8 |
| 15 | 1-(4′-isopropylphenyl)-3-phenylpropane-1,3-dione | 63260-25-9 |
| 16 | 4-isopropylbenzyl salicylate | 94134-93-7 |
| 17 | 3-imidazol-4-ylacrylic acid and the ethyl ester thereof | 104-98-3 |
| 18 | ethyl 2-cyano-3,3-diphenylacrylate | 5232-99-5 |
| 19 | 2′-ethylhexyl 2-cyano-3,3-diphenylacrylate | 6197-30-4 |
| 20 | menthyl o-aminobenzoate or | 134-09-8 |
| | 5-methyl-2-(1-methylethyl)-2-aminobenzoate | |
| 21 | glyceryl p-aminobenzoate or | 136-44-7 |
| | 1-glyceryl 4-aminobenzoate | |
| 22 | 2,2′-dihydroxy-4-methoxybenzophenone (dioxybenzone) | 131-53-3 |
| 23 | 2-hydroxy-4-methoxy-4-methylbenzophenone | 1641-17-4 |
| | (mexenone) | |
| 24 | triethanolamine salicylate | 2174-16-5 |
| 25 | dimethoxyphenylglyoxalic acid or | 4732-70-1 |
| | sodium 3,4-dimethoxyphenylglyoxalate | |
| 26 | 3-(4′-sulfobenzylidene)bornan-2-one and salts thereof | 56039-58-8 |
| 27 | 4-tert-butyl-4′-methoxydibenzoylmethane | 70356-09-1 |
| 28 | 2,2′,4,4′-tetrahydroxybenzophenone | 131-55-5 |
| 29 | 2,2′-methylenebis[6-(2H-benzotriazol-2-yl)-4-(1,1,3,3- | 103597-45-1 |
| | tetramethylbutyl)phenol] | |
| 30 | 2,2′-(1,4-phenylene)bis-1H-benzimidazole-4,6- | 180898-37-7 |
| | disulfonic acid, sodium salt | |
| 31 | 2,4-bis-[4-(2-ethylhexyloxy)-2-hydroxy]phenyl- | 187393-00-6 |
| | 6-(4-methoxyphenyl)-(1,3,5)-triazine | |
| 32 | 3-(4-methylbenzylidene)camphor | 36861-47-9 |
| 33 | polyethoxyethyl 4-bis(polyethoxy)paraaminobenzoate | 113010-52-9 |
| 34 | 2,4-dihydroxybenzophenone | 131-56-6 |
| 35 | 2,2′-dihydroxy-4,4′-dimethoxybenzophenone-5,5′- | 3121-60-6 |
| | disodium sulfonate | |
| 36 | benzoic acid 2-[4-(diethylamino)-2-hydroxybenzoyl]hexyl ester | 302776-68-7 |
| 37 | 2-(2H-benzotriazol-2-yl)-4-methyl-6-[2-methyl-3-[1,3,3,3- | 155633-54-8 |
| | tetramethyl-1-[(trimethylsily)oxy]disiloxanyl]propyl]phenol | |
| 38 | 1,1-[(2,2′-dimethylpropoxy)carbonyl]-4,4-diphenyl-1,3-butadiene | 363602-15-7 |

In addition to the two aforementioned groups of primary light stabilizers, it is also possible to use secondary light stabilizers of the antioxidant type, which stop the photochemical reaction chain which is triggered when UV radiation penetrates into the skin. Typical examples thereof are tocopherols (vitamin E) and oil-soluble ascorbic acid derivatives (vitamin C).

According to the invention, it is possible to use suitable derivatives (salts, esters, sugars, nucleotides, nucleosides, peptides and lipids) of the compounds mentioned as effect substances.

Further preferred are what are called peroxide decomposers, i.e. compounds which are capable of decomposing peroxides, more preferably lipid peroxides. These are understood to mean organic substances, for example 5-pyrimidinol derivatives and 3-pyridinol derivatives and probucol.

In addition, the peroxide decomposers mentioned are preferably the substances described in patent applications WO-A-02/07698 and WO-A03/059312, the content of which is hereby explicitly incorporated by reference, preferably the boron-comprising or nitrogen-comprising compounds described therein, which can reduce peroxides or hydroperoxides to the corresponding alcohols without forming free-radical conversion stages. In addition, it is possible to use sterically hindered amines for this purpose.

A further group is that of antiirritants, which have an inflammation-inhibiting action on skin damaged by UV light. Such substances are, for example, bisabolol, phytol and phytantriol.

A further group of effect substances is that of active ingredients which can be used in crop protection, for example herbicides, insecticides and fungicides.

The following list of insecticides shows possible active crop protection ingredients, but no restriction thereto is intended:

A.1. organo(thio)phosphates: azinphos-methyl, chlorpyrifos, chlorpyrifos-methyl, chlorfenvinphos, diazinon, disulfoton, ethion, fenitrothion, fenthion, isoxathion, malathion, methidathion, methyl-parathion, oxydemeton-methyl, paraoxon, parathion, phenthoate, phosalone, phosmet, phosphamidon, phorate, phoxim, pirimiphos-methyl, profenofos, prothiofos, sulprophos, tetrachlorvinphos, terbufos, triazophos, trichlorfon;

A.2. carbamates: alanycarb, bendiocarb, benfuracarb, carbaryl, carbofuran, carbosulfan, fenoxycarb, furathiocarb, methiocarb, methomyl, oxamyl, pirimicarb, thiodicarb, triazamate;

A.3. pyrethroids: allethrin, bifenthrin, cyfluthrin, cyhalothrin, cyphenothrin, cypermethrin, alpha-cypermethrin, beta-cypermethrin, zeta-cypermethrin, deltamethrin, esfenvalerate, etofenprox, fenpropathrin, fenvalerate, imiprothrin, lambda-cyhalothrin, permethrin, prallethrin, pyrethrin I and II, resmethrin, silafluofen, tau-fluvalinate, tefluthrin, tetramethrin, tralomethrin, transfluthrin;

A.4. growth regulators: a) chitin synthesis inhibitors: benzoylureas: chlorfluazuron, cyramazin, diflubenzuron, flucycloxuron, flufenoxuron, hexaflumuron, lufenuron, novaluron, teflubenzuron, triflumuron; buprofezin, diofenolan, hexythiazox, etoxazole, clofentazine; b) ecdysone antagonists: halofenozide, methoxyfenozide, tebufenozide, azadirachtin; c) juvenoids: pyriproxyfen, methoprene, fenoxycarb; d) lipid biosynthesis inhibitors: spirodiclofen, spiromesifen, a tetronic acid derivative of formula D1

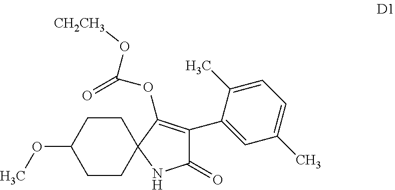

A.5. nicotine receptor agonists/antagonists: clothianidin, dinotefuran, thiacloprid;

A.6. GABA antagonists: acetoprole, endosulfan, ethiprole, fipronil, vaniliprole;

A.7. macrolide insecticides: abamectin, emamectin, milbemectin, lepimectin, spinosad;

A.8. MET1 I acaricides: fenazaquin, pyridaben, tebufenpyrad, tolfenpyrad;

A.9. MET1 II and III compounds: acequinocyl, fluacyprim, hydramethylnon;

A.10. uncoupler compounds: chlorfenapyr;

A.11. inhibitors of oxidative phosphorylation: cyhexatin, diafenthiuron, fenbutatin oxide, propargite;

A.12. ecdysis-inhibiting compounds: cryomazine;

A.13. inhibitors of the mixed function oxidase: piperonyl butoxide;

A.14. sodium channel blockers: indoxacarb, metaflumizone;

A.15. various: benclothiaz, bifenazate, flonicamid, pyridalyl, pymetrozine, sulfur, thiocyclam and aminoisothiazole compounds of the formula D2

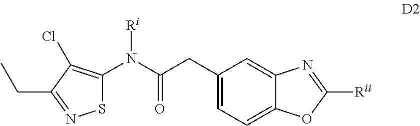

where Rⁱ is —CH₂OCH₂CH₃ or H and Rⁱⁱ is CF₂CF₂CF₃ or CH₂CH(CH₃)₃, anthranilamide compounds of the formula D3

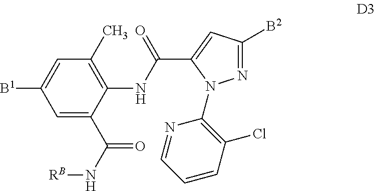

where B¹ is hydrogen or chlorine, B² is bromine or CF₃ and R^{B} is CH₃ or CH(CH₃)₂, and malononitrile compounds as described in JP 2002 284608, WO 02/189579, WO 02/190320, WO 02/190321, WO 04/06677, WO 04/120399 or JP 2004 99597, N-R′-2,2-dihalo-1-R″-cyclopropanecarboxamide-2-(2,6-dichloro-α,α,α,α-trifluoro-p-tolyβhydrazone or N-R′-2,2-di(R′″)propionamide-2-(2,6-dichloro-α,α,α,α-trifluoro-p-toly)hydrazone in which R′ is methyl or ethyl, halo is chlorine or bromine, R″ is hydrogen or methyl and R′″ is methyl or ethyl.

The list of fungicides below shows possible active ingredients, but no restriction thereto is intended:

1. Strobilurins

azoxystrobin, dimoxystrobin, enestrostrobin, fluoxastrobin, kresoxim-methyl, metomino-strobin, picoxystrobin, pyraclostrobin, trifloxystrobin, orysastrobin, methyl (2-chloro-5-[1-(3-methylbenzyloxyimino)ethyl]benzyl)carbamate, methyl (2-chloro-5-[1-6-methylpyridin-2-ylmethoxyimino)ethyl]benzyl)carbamate, methyl 2-(ortho-(2,5-dimethylphenyloxymethylene)phenyl)-3-methoxyacrylate.

2. Carboxamides,

carboxanilides: benalaxyl, benodanil, boscalid, carboxin, mepronil, fenfuram, fenhex-amid, flutolanil, furametpyr, metalaxyl, ofurace, oxadixyl, oxycarboxin, penthiopyrad, thifluzamide, tiadinil, N-(4′-bromobiphenyl-2-yl)-4-difluoromethyl-2-methylthiazole-5-carboxamide, N-(4′-trifluoromethylbiphenyl-2-yl)-4-difluoro-2-methyltriazole-5-carboxamide, N-(4′-chloro-3′-fluorobiphenyl-2-yl)-4-difluoro-2-methyltriazole-5-carboxamide, N-(3′,4′-dichloro-4-fluorobiphenyl-2-yl)-3-difluoro-1-methylpyrazole-4-carboxamide;

carboxylic acid morpholides: dimethomorph, flumorph;

benzamides: flumetover, fluopicolide (picobenzamid), zoxamide;

other carboxamides: carpropamid, diclocymet, mandipropamid, N-(2-(4-[3-(4-chloro-phenyl)prop-2-ynyloxy]-3-methoxyphenyl)ethyl)-2-methanesulfonylamino-3-methyl-butyramide, N-(2-(4-[3-(4-chlorophenyl)prop-2-ynyloxy]-3-methoxyphenyl)ethyl)-2-ethanesulfonylamino-3-methylbutyramide;

3. Azoles

triazoles: bitertanol, bromuconazole, cyproconazole, difenoconazole, diniconazole, enilconazole, epoxiconazole, fenbuconazole, flusilazole, fluquinconazole, flutriafol, hexaconazole, imibenconazole, ipconazole, metconazole, myclobutanil, penconazole; propiconazole, prothioconazole, simeconazole, tebuconazole, tetraconazole, triadimenol, triadimefon, triticonazole;

imidazoles: cyazofamid, imazalil, pefurazoate, prochloraz, triflumizole;

benzimidazoles: benomyl, carbendazim, fuberidazole, thiabendazole;

others: ethaboxam, etridiazole, hymexazole;

4. Nitrogen-containing Heterocyclyl Compounds

pyridines: fluazinam, pyrifenox, 3-[5-(4-chlorophenyl)-2,3-dimethylisoxazolidin-3-yl]-pyridine;

pyrimidines: bupirimate, cyprodinil, ferimzone, fenarimol, mepanipyrim, nuarimol, pyrimethanil;

piperazines: triforine;

pyrroles: fludioxonil, fenpiclonil;

morpholines: aldimorph, dodemorph, fenpropimorph, tridemorph;

dicarboximides: iprodione, procymidone, vinclozolin;

others: acibenzolar-S-methyl, anilazine, captan, captafol, dazomet, diclomezine, fenoxanil, folpet, fenpropidin, famoxadone, fenamidone, octhilinone, probenazole, proquinazid, quinoxyfen, tricyclazole, 5-chloro-7-(4-methylpiperidin-1-yl)-6-(2,4,6-tri-fluorophenyl)-[1,2,4]triazolo[1,5-a]pyrimidine, 2-butoxy-6-iodo-3-propylchromen-4-one, N,N-dimethyl-3-(3-bromo-6-fluoro-2-methylindole-1-sulfonyl)-[1,2,4]triazole-1-sulfonamide;

5. Carbamates and Dithiocarbamates

carbamates: diethofencarb, flubenthiavalicarb, iprovalicarb, propamocarb, methyl 3-(4-chlorophenyI)-3-(2-isopropoxycarbonylamino-3-methylbutyrylamino)propionate, 4-fluorophenyl N-(1-(1-(4-cyanophenyl)ethanesulfonyl)but-2-yl)carbamate;

6. Other Fungicides

organometallic compounds: fentin salts;

sulfur-containing heterocyclyl compounds: isoprothiolane, dithianon;

organophosphorus compounds: edifenphos, fosetyl, fosetyl-aluminum, iprobenfos, pyrazophos, tolclofos-methyl, phosphorous acid and its salts;

organochlorine compounds: thiophanate-methyl, chlorothalonil, dichlofluanid, tolylfluanid, flusulfamide, phthalide, hexachlorobenzene, pencycuron, quintozene;

nitrophenyl derivatives: binapacryl, dinocap, dinobuton;

others: spiroxamine, cyflufenamid, cymoxanil, metrafenone.

The list of herbicides below shows possible active ingredients, but no restriction thereto is intended:

compounds which inhibit the biosynthesis of lipids, for example chlorazifop, clodinafop, clofop, cyhalofop, ciclofop, fenoxaprop, fenoxaprop-p, fenthiaprop, fluazifop, fluazifop-P, haloxyfop, haloxyfop-P, isoxapyrifop, metamifop, propaquizafop, quizalofop, quizalofop-P, trifop, or esters thereof, butroxydim, cycloxydim, profoxydim, sethoxydim, tepraloxydim, tralkoxydim, butylate, cycloate, diallate, dimepiperate, EPTC, esprocarb, ethiolate, isopolinate, methiobencarb, molinate, orbencarb, pebulate, prosulfocarb, sulfallate, thiobencarb, thiocarbazil, triallate, vernolate, benfuresate, ethofumesate and bensulide;

ALS inhibitors such as amidosulfuron, azimsulfuron, bensulfuron, chlorimuron, chlorsulfuron, cinosulfuron, cyclosulfamuron, ethametsulfuron, ethoxysulfuron, flazasulfuron, flupyrsulfuron, foramsulfuron, halosulfuron, imazosulfuron, iodosulfuron, mesosulfuron, metsulfuron, nicosulfuron, oxasulfuron, primisulfuron, prosulfuron, pyrazosulfuron, rimsulfuron, sulfometuron, sulfosulfuron, thifensulfuron, triasulfuron, tribenuron, trifloxysulfuron, triflusulfuron, tritosulfuron, imazamethabenz, imazamox, imazapic, imazapyr, imazaquin, imazethapyr, cloransulam, diclosulam, florasulam, flumetsulam, metosulam, penoxsulam, bispyribac, pyriminobac, propoxycarbazone, flucarbazone, pyribenzoxim, pyriftalid and pyrithiobac; if the pH is <8;

compounds which inhibit photosynthesis, such as atraton, atrazine, ametryne, aziprotryne, cyanazine, cyanatryn, chlorazine, cyprazine, desmetryne, dimethametryne, dipropetryn, eglinazine, ipazine, mesoprazine, methometon, methoprotryne, procyazine, proglinazine, prometon, prometryne, propazine, sebuthylazine, secbumeton, simazine, simeton, simetryne, terbumeton, terbuthylazine and terbutryne;

protoporphyrinogen-IX oxidase inhibitors such as acifluorfen, bifenox, chlomethoxyfen, chlormitrofen, ethoxyfen, fluorodifen, fluoroglycofen, fluoronitrofen, fomesafen, furyloxyfen, halosafen, lactofen, nitrofen, nitrofluorfen, oxyfluorfen, fluazolate, pyraflufen, cinidon-ethyl, flumiclorac, flumioxazin, flumipropyn, fluthiacet, thidiazimin, oxadiazon, oxadiargyl, azafenidin, carfentrazone, sulfentrazone, pentoxazone, benzfendizone, butafenacil, pyraclonil, profluazol, flufenpyr, flupropacil, nipyraclofen and etnipromid;

herbicides such as metflurazon, norflurazon, flufenican, diflufenican, picolinafen, beflubutamid, fluridone, flurochloridone, flurtamone, mesotrione, sulcotrione, isoxachlortole, isoxaflutole, benzofenap, pyrazolynate, pyrazoxyfen, benzobicyclon, amitrole, clomazone, aclonifen, 4-(3-trifluoromethylphenoxy)-2-(4-trifluoromethylphenyl)pyrimidine and 3-heterocyclyl-substituted benzoyl derivatives of the formula (cf. WO-A-96/26202, WO-A-97/41116, WO-A-97/41117 and WO-A-97/41118)

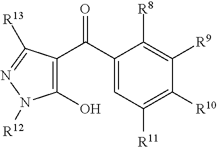

in which the substituents R⁸ to R¹³ are each defined as follows: R8, R¹⁰ are hydrogen, halogen, C₁-C₅-alkyl, C₁-C₅-haloalkyl, C₁-C₅-alkoxy, haloalkoxy, C₁-C₅-alkylthio, C₁-C₅-alkylsulfinyl or C₁-C₅-alkylsulfonyl; R⁹ is a heterocyclic radical from the group consisting of thiazol-2-yl, thiazol-4-yl, thiazol-5-yl, isoxazol-3-yl, isoxazol-4-yl, isoxazol-5-yl, 4,5-dihydroisoxazol-3-yl, 4,5-dihydroisoxazol-4-yl and 4,5-dihydroisoxazol-5-yl, where the radicals mentioned may bear one or more substituents; for example, they may be mono-, di-, tri- or tetrasubstituted by halogen, C₁-C₄-alkyl, C₁-C₄-alkoxy, C₁-C₄-haloalkyl, C₁-C₄-halo-alkoxy or C₁-C₄-alkylthio;

R¹¹=hydrogen, halogen or C₁-C₅-alkyl;
R¹²C₁-C₆-alkyl;
R¹³=hydrogen or C₁-C₆-alkyl if the pH is <8;
mitosis inhibitors, such as benfluralin, butralin, dinitramine, ethalfluralin, fluchloralin, isopropalin, methalpropalin, nitralin, oryzalin, pendimethalin, prodiamine, profluralin, trifluralin, amiprofos-methyl, butamifos, dithiopyr, thiazopyr, propyzamide, chlorthal, carbetamide, chlorpropham and propham;
VLCFA inhibitors, such as acetochlor, alachlor, butachlor, butenachlor, delachlor, diethatyl, dimethachlor, dimethenamid, dimethenamid-P, metazachlor, metolachlor, S-metolachlor, pretilachlor, propisochlor, prynachlor, terbuchlor, thenylchlor, xylachlor, CDEA, epronaz, diphenamid, napropamide, naproanilide, pethoxamid, flufenacet, mefenacet, fentrazamide, anilofos, piperophos, cafenstrole, indanofan and tridiphane; inhibitors of the biosynthesis of cellulose, such as dichlobenil, chlorthiamid, isoxaben and flupoxam;

herbicides, such as dinofenate, dinoprop, dinosam, dinoseb, dinoterb, DNOC, etinofen and medinoterb;

also: benzoylprop, flamprop, flamprop-M, bromobutide, chlorflurenol, cinmethylin, methyldymron, etobenzanid, pyributicarb, oxaziclomefone, triaziflam and methyl bromide.

Active ingredients used in crop protection can also be used to control pests (for example cockroaches, ants, termites inter alia) in an urban situation (for example residential developments, domestic and garden sectors, restaurants, car parks, industrial areas inter alia) and are a further group of suitable effect substances specifically for these applications.

It is also possible to formulate active ingredients for controlling pests from the field of vertebrates (for example rats, mice inter alia) with the process according to the invention, and to employ the resulting active ingredient formulations for corresponding pest control in agriculture and in an urban situation.

Additionally suitable are active ingredients for pharmaceutical use, especially those for oral administration. The process according to the invention is in principle applicable to a multitude of active ingredients irrespective of the medical indication.

Particular mention should be made of water-soluble active ingredients for pharmaceutical use, especially those for oral administration. This relates both to prescription-only and over the counter active ingredients. The invention is in principle applicable to a multitude of active ingredients irrespective of the medical indication.

Nonlimiting examples of suitable sparingly water-soluble active pharmaceutical ingredients are specified in the following table:

| Active | Empirical | Solubility in water | |
|---|---|---|---|
| ingredient | formula | [g/l] | |
| Felodipine | C₁₈H₁₉Cl₂NO₄ | 4.53E−03 | (22° C.) |
| Indomethacin | C₁₉H₁₆ClNO₄ | 1.4E−02 | (25° C.) |
| Piroxicam | C₁₅H₁₃N₃O₄S | 2.3E−02 | (RT) |
| Carbamazipine | C₁₅H₁₂N₂O | 9.451E−01 | (RT) |
| 17-β-Estradiol | C₁₈H₂₄O₂ | 1.836E−05 | (25° C.) |
| Clotrimazole | C₂₂H₁₇ClN₂ | <1.0E−02 | (25° C.) |
| Ketoconazole | C₂₆H₂₈Cl₂N₄O₄ | 8.0E−02 | (37° C.) |
| Cinnarizine | C₂₆H₂₈N₂ | 7.5E−01 | |
| Griseofulvin | C₁₇H₁₇ClO₆ | 3.685E−05 | (25° C.) |
| Ibuprofen | C₁₃H₁₈O₂ | 2.1E−02 | (25° C.) |

Examples of water-soluble active pharmaceutical ingredients are especially active cough-inducing and mucotic ingredients, for example guaiacol glycol ether (also known as guaifenesin) and derivatives thereof.

Further preferred active pharmaceutical ingredients are antibodies and other proteins used in pharmacy, for example enzymes or peptides, or nucleic acids.

(iv) Active Ingredient Release from the Formulations

The active ingredients can be released from the formulations produced by the process according to the invention by desorption into suitable solvents, by the degradation of the fibrous sheetlike structures by hydrolysis, oxidation, or biologically by means of enzymes, for example proteases, or whole microorganisms, or by dissolution of the fibrous sheetlike structure by means of suitable solvents, and by diffusion of the active ingredient to the fibrous surface. Suitable solvents for the desorption are all solvents or solvent mixtures in which the active ingredient can be dissolved. Solvents which can dissolve the fibrous sheetlike structures may be solvents only suitable for the carrier polymer system, or suitable for the carrier polymer system and the active ingredient.

A particular advantage of this invention is the delayed active ingredient release, for which chemical factors, for example composition of the carrier, can be combined with a defined configuration of the nano- and mesofibers (controlled specific surface area). This allows the release to be controlled much more precisely.

The kinetics and the profile of the release of the effect substance molecules can, for example, be controlled:
(i) by the loading density of the carrier polymer with active ingredients;
(ii) by specific surface area of the fibers (i.e. diameter);
(iii) by the use of a polymer mixture of at least two polymers as the carrier polymer, which do not have equally good solubility in the same solvent. In other words, by variation of the ratio of the soluble and sparingly soluble or insoluble polymer in the particular solvent;
(iv) by use of a biodegradable synthetic polymer or of a biopolymer as the carrier;
(v) by variation of the ratio in the mixture of a non-biodegradable polymer with a biodegradable polymer;
(vi) by variation of the chemical structure of the non-biodegradable polymer (e.g.

water-soluble/water-insoluble) in the mixture of a non-biodegradable polymer with a biodegradable polymer;
(vii) by variation of the ratio in the mixture of a non-biodegradable polymer with a biodegradable polymer;
(viii) by the use of a polymer mixture of at least two non-biodegradable polymers as the carrier polymer, which do not have equally good solubility in the release medium, and a biodegradable polymer;
(ix) by use of a mixture of immiscible polymers, the fibers having chemical structuring (phase separation);
(x) by use of homopolymers, copolymers or polymer blends which have at least one phase with Tg below the use temperature;
(xi) by physical structuring in the form of porosity and/or surface roughness (topography); and
(xii) combination of the above measures.

The invention further provides for the use of the fibrous sheetlike structures produced using the polymers described for storage, for transport or for release of active ingredients in cosmetic products, human and animal pharmaceutical products, crop protection products, foods and animal feeds. The fibrous sheetlike structures further serve to protect the packaged active ingredients from environmental influences, for example oxidative processes or UV radiation, or from destruction by reacting with other constituents of the products or from biodegradation by enzymes (e.g. proteases) or microorganisms. The active ingredient can be released from the fibrous sheetlike structures by desorption, biodegradation, controlled release or slow release, or a combination of these measures.

By variation of the amino acid sequence of the amphiphilic self-assembly proteins described, or fusion with additional protein or peptide sequences, it is possible to generate structures which specifically recognize particular surfaces, for example skin, hair, leaves, roots, and are recognized and bound by these surfaces or the receptors present.

It is thus possible to bring the active ingredients formulated with the amphiphilic self-assembly proteins described more effectively to the desired site of action, or to improve the active ingredient absorption.

In addition, it is possible by variation of the amino acid sequence of the amphiphilic self-assembly proteins described for the active ingredient formulation, or fusion with additional protein or peptide sequences, to direct active ingredients in a controlled manner to desired sites of action, in order thus to achieve, for example, higher specificity, lower active ingredient consumption or active ingredient dose, faster or more rapid action.

Experimental Section

General Section:

a) Electrospinning Processes

The electrospinning apparatus suitable for performance of the process according to the invention comprises a syringe provided at its tip with a capillary nozzle connected to one pole of a voltage source, to accommodate the inventive formulation. Opposite the exit of the capillary nozzle is arranged, at a distance of about 20 cm, a square counterelectrode connected to the other pole of the voltage source, which functions as the collector for the fibers formed. During the operation of the apparatus, a voltage between 15 kV and 35 kV is established at the electrodes and the formulation is discharged through the capillary nozzle of the syringe under a low pressure. The electrostatic charging of the formulation caused by the strong electrical field of 0.9 to 2 kV/cm results in a material flow directed toward the counterelectrode, which solidifies on the way to the counterelectrode to form fibers, as a result of which fibers with diameters in the micro- and nanometer range are deposited at the counterelectrode.

A further possible apparatus for performance of the process according to the invention comprises a roller which rotates within a vessel containing spinning solution. The roller may be smooth or have physical structuring, for example needles or grooves. On each rotation of the roller, the spinning solution gets into the strong electrical field, and several material streams are formed. The counterelectrode is above the spinning electrode. The fibers are deposited on a carrier nonwoven, e.g. polypropylene. For example, it is possible to use a Nanospider apparatus from Elmarco. The voltage is about 82 kV at an electrode distance of 18 cm. The temperature is about 23° C. and the relative air humidity 35%. A serrated electrode is used for spinning. In order to achieve a sheetlike protein structure of maximum thickness (e.g. protein films, protein fibers, protein nonwovens), the carrier nonwoven is left stationary. Alternatively, the carrier nonwoven can also be moved with an advance rate to achieve relatively thin sheetlike protein structure layers in a defined manner.

b) Sample Preparation for WAXS Analysis

The samples were prepared between two adhesive tape strips (commercial product from Scotch), and the transmission thereof was measured.

c) Active Ingredient Release Tests

The release of the active ingredients from the fibrous sheetlike structures was examined by the long-time encapsulation analysis method. In this method, the encapsulated active ingredients are made up in a defined concentration below the solubility limit of the active ingredient in demineralized (DM) water. The samples are kept stirred over a period of minutes up to several weeks. At logarythmically graduated time intervals, a sample is taken each time and the free active ingredient present therein is analyzed by chromatography. On the basis of the calibration of the active ingredient carried out beforehand, the amount released can thus be determined.

Active ingredient release tests with protein-containing formulations can also be carried out in two further experimental variants:

Active ingredient formulations to be taken perorally (for example clotrimazole (pressed to tablets)) can be analyzed in synthetic gastric juice (0.1 g of NaCl; 0.16 g of pepsin; make up 0.35 ml of HCl to 50 ml, pH 1-2) and synthetic intestinal juice (dissolve 3.4 g of KH₂PO₄ in 12.5 ml of water+make up 3.85 ml of 0.2N NaOH to 25 ml+make up 0.5 g of pancreatin to 50 ml, pH 6.8), in order to simulate the release of active ingredient under proteolytically active conditions in the digestive tract. Controlled tests (without proteases) were effected in 5 mM potassium phosphate buffer (pH 8.0), and only a small release of active ingredient should be observed under these conditions. 20 ml of the particular digestive juice or buffer were added per tablet, and the mixtures were incubated with slight agitation at 37° C. and 80 rpm. At different times, 500 μl of sample in each case were taken for an active ingredient quantification by means of HPLC or a photometer. In order also to detect active ingredient aggregates formed after the release in the case of sparingly water-soluble active ingredients, for example clotrimazole, the absorption photometry quantification was performed after extraction with THF (3 ml of supernatant+3 ml of THF+spatula-tip of NaCl, vigorous vortexing, 1 min at 15 000×g, analyze upper phase, dilute if appropriate).

In the case of other active ingredients (active pharmaceutical ingredients not taken perorally or other active ingredients), for example Uvinul A+ and metazachlor, the release analysis can be effected by admixing defined amounts of sheetlike protein-active ingredient structures with unspecific proteinase K solution. The sheetlike protein-active ingredient structures were incubated in 0.25-0.5% [w/v] proteinase K (Roche, Germany; dissolved in 5 mM potassium phosphate buffer) with agitation at 120-150 rpm. At different times, the still-intact sheetlike protein-active ingredient structures were removed by centrifugation, the supernatants were admixed with a 4-5-fold excess of THF and the active ingredient content was subsequently determined by absorption photometry. In all experiments, the amounts of active ingredient released were determined after comparison with an active ingredient-specific calibration series.

### EXAMPLE 1

### Production and Properties of the Composite Fibers from PVP and Epoxiconazole

To produce composite fibers, polymer solutions were produced from poly(1-vinyl-2-pyrrolidinone) Kollidon K-90 (PVP) (Mw=1 100 000 g/mol, Tg=180° C., BASF SE) and epoxiconazole fungicide (1-{[3-(2-chlorophenyl)-2-(4-fluorophenyl)oxiran-2-yl]methyl}-1H-1,2,4-triazole) in an ethanol/water mixture (90:10) and spun to fibers. This involves spinning the solutions with a spinning system under voltages between 35 and 45 kV.

The starting weights are listed in the following table:

| | | Active |
|---|---|---|
| | | ingredient |
| | | content, % by |
| Material | Mass, g | weight |
| Ethanol | 7.104 | |
| Water | 0.998 | |
| PVP | 0.486 | |
| Epoxiconazole | 0.0486 | 9.1 |
| ″ | 0.0972 | 16.7 |
| ″ | 0.146 | 23.1 |
| ″ | 0.194 | 28.6 |
| ″ | 0.243 | 33.3 |

The concentration figures of the epoxiconazole active ingredient are based on total solids (PVP+active ingredient). The concentration of the carrier polymer is based on the total mass of solvent and polymer before the addition of the active ingredient.

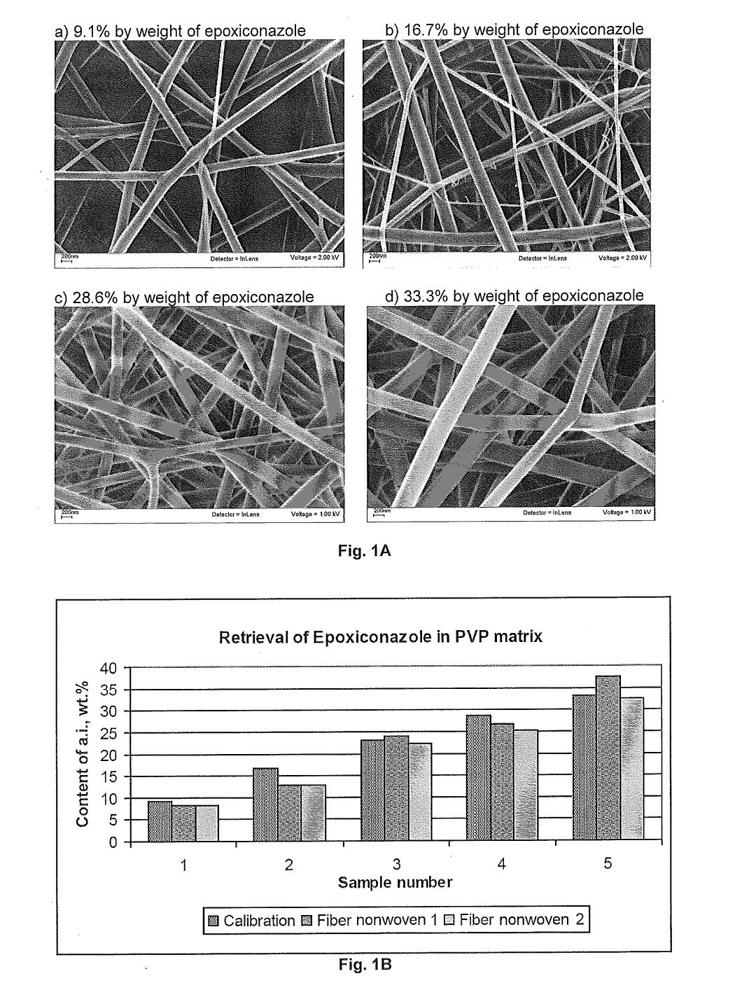

FIG. 1A shows the fiber morphology as a function of the active ingredient contents.

In order to be able to make statements about the proportion of epoxiconazole in the fibers, a calibration was carried out at the start. For this purpose, solutions of epoxiconazole with concentrations of 10 to 40% by weight (based on the solids content) and 5.7% by weight of PVP (based on the total weight of the formulation before the addition of the active ingredient) in ethanol/water (90/10) were prepared and applied to an Si wafer, and the transmission thereof was analyzed by means of IR spectroscopy. The ratio of the specific bands of PVP and epoxiconazole was evaluated and used to draw up a calibration plot.

The active ingredient-containing fibrous sheetlike structures produced were likewise dissolved in the ethanol/water mixture and applied as a film to Si wafers analogously to the calibration samples, and analyzed by IR spectroscopy, and the calibration plot was used to determine the concentrations of epoxiconazole. The calibration values together with the findings from fibrous sheetlike structures are shown in FIG. 1B.

The graph shows that, after the fibers have been spun, approximately the same amount of epoxiconazole as used is still present. Several measurements show that the result is reproducible.

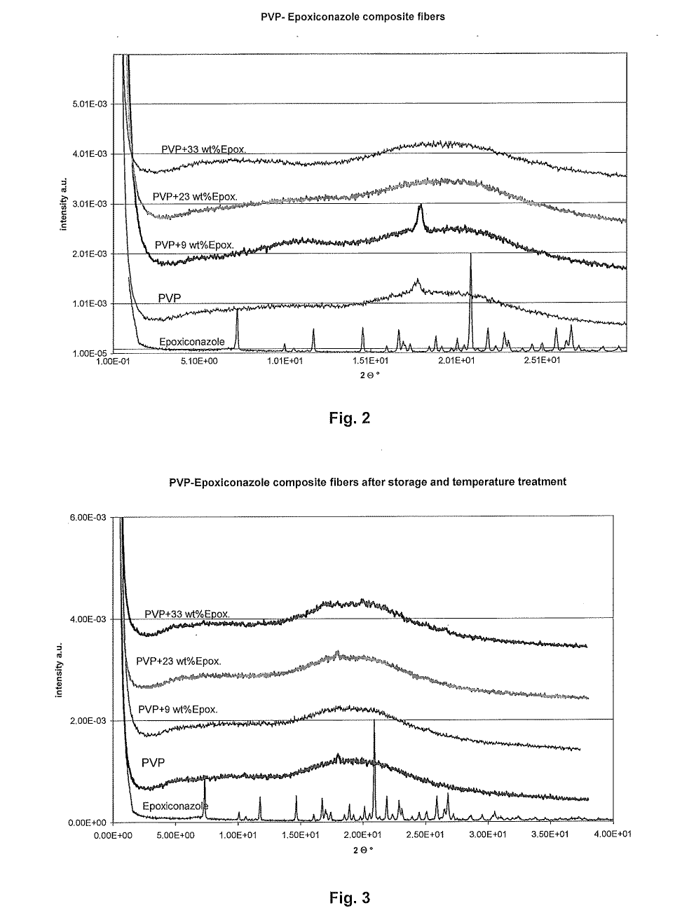

The epoxiconazole active ingredient is present in an amorphous state in the fibrous sheetlike structure. This is confirmed by the wide-angle x-ray scattering analyses (WAXS), which were carried out with a Bruker D5005 diffractometer (monochromatized Cu-Kα radiation) in transmission. Results of the WAXS analyses on freshly prepared fibrous sheetlike structures composed of PVP-epoxiconazole are shown in FIG. 2.

This involved enclosing the samples on or between two adhesive tape strips. The sharp peak at about 2Θ=18° is an impurity, since this peak is already observed in the pure PVP and cannot be assigned to the epoxiconazole.

In order to test the storage stability of these fibrous sheetlike structures, the samples were stored at +40° C., −10° C. and 0° C. for 24 h in each case and at 20° C. for 72 h, and then analyzed again by means of wide-angle x-ray scattering

The results of the WAXS analyses on fibrous sheetlike structures composed of PVP-epoxiconazole stored at different temperatures are shown in FIG. 3.

FIG. 3 shows clearly that the formulations are storage-stable—there is no change in the amorphous morphology of active ingredient in the course of storage at different temperatures.

### EXAMPLE 2

### Production and Properties of the Composite Fibers Formed by PVP and Beta-carotene.

β-Carotene is used to color fatty foods such as butter, margarine, cheese, mayonnaise and—in water-dispersible form—also water-containing foods, for example fruit drinks, puddings, confectionary. β-carotene is also used as a dye for cosmetics and as an animal feed additive. To produce composite fibers, polymer solutions were produced from poly(1-vinyl-2-pyrrolidinone) Kollidon K-90 (PVP) (Mw=1 100 000 g/mol, Tg=180° C., BASF SE) and the β-carotene dye in chloroform and spun to fibers. For this purpose, the solutions were spun with a syringe system under voltages between 40 and 45 kV. In addition, 0.5% by weight, based on the overall formulation, of benzyltributylammonium bromide was added thereto in order to increase the electrical conductivity of the solution. This has a positive effect on the fiber morphology and diameter distribution: fewer beads form and the fiber diameter distribution becomes narrower.

The starting weights are listed in the following table:

| | | Active |
|---|---|---|
| | | ingredient |
| | | content, % by |
| Material | Mass, g | weight |
| Chloroform | 74 | |
| PVP | 4.41 | |
| β-Carotene | 0.441 | 9.1 |
| ″ | 0.882 | 16.7 |
| ″ | 1.323 | 23.1 |
| ″ | 1.764 | 28.6 |
| ″ | 2.205 | 33.3 |

The concentration figures for the β-carotene effect substance are based on the total mass of PVP and effect substance. The concentration of the carrier polymer is based on the total mass of solvent and polymer.

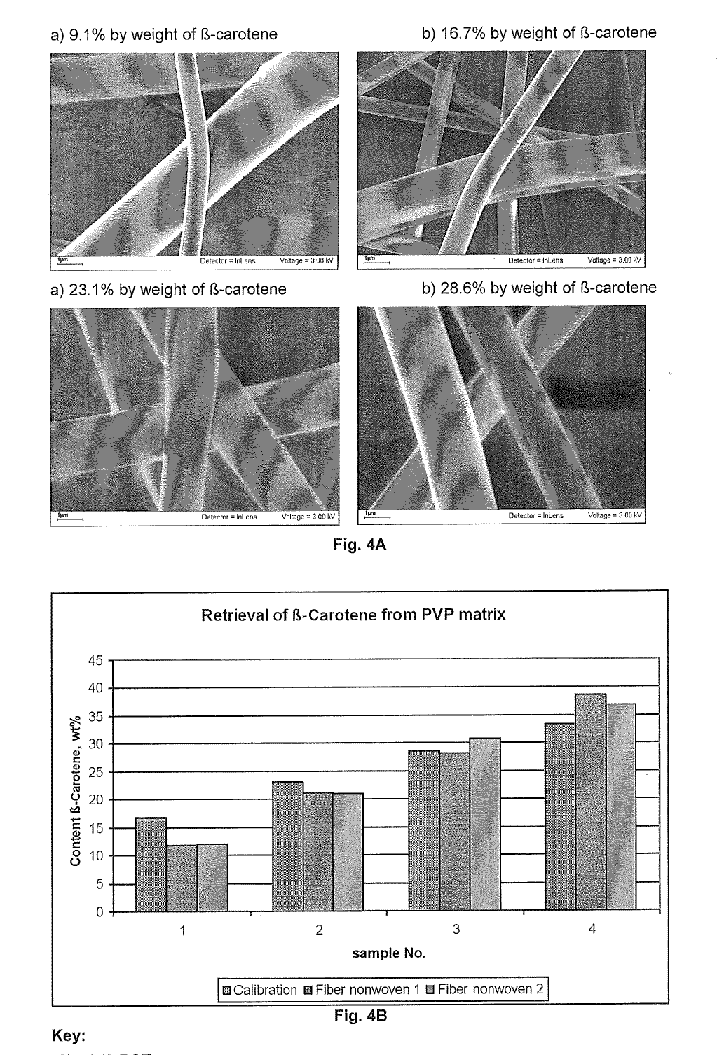

FIG. 4A shows the fiber morphology as a function of the effect content.

In order to be able to make statements about the proportion of β-carotene in the fibers, a calibration was carried out at the start. For this purpose, solutions of β-carotene with concentrations of 10 to 40% by weight (based on the solids content) and 6% by weight of PVP (based on the total weight of the formulation before the addition of the active ingredient) were prepared in chloroform and applied to an Si wafer, and the transmission thereof was analyzed by means of IR spectroscopy. The ratio of the specific bands of PVP and β-carotene was evaluated and used to draw up a calibration plot.

The effect substance-containing fibrous sheetlike structures produced were dissolved in chloroform and, like the calibration samples, applied to Si wafers as a film and analyzed by IR spectroscopy, and the β-carotene concentrations were evaluated using the calibration plot. The calibration values together with the findings from fibrous sheetlike structures are shown in FIG. 4B.

The diagram of FIG. 4B shows that, after the spinning, the fibers still have about the amount of β-carotene used. Several measurements show that the result is reproducible.

The β-carotene effect substance is present in an amorphous state. This is shown by the wide-angle x-ray scattering analyses (WAXS), which were carried out with a Bruker D5005 diffractometer (monochromatized Cu-Kα radiation) in transmission.

The samples were enclosed between two adhesive tape strips.

FIG. 5 shows the results of the WAXS analyses on freshly prepared fibrous sheetlike structures composed of PVP-β-carotene.

In order to test the storage stability of these fibrous sheetlike structures, the samples were stored at +40° C., −10° C. and 0° C. for 24 h in each case, and at 20° C. for at least 72 h, and then analyzed again by means of wide-angle x-ray scattering

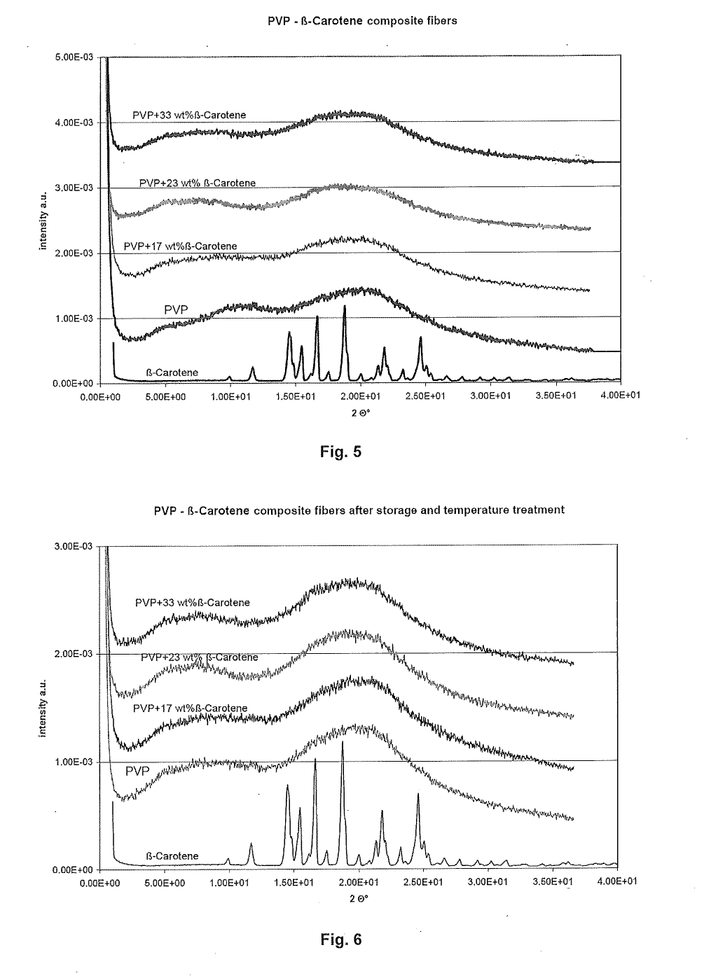

FIG. 6 shows results of the WAXS analyzes on fibrous sheetlike structures composed of PVP-β-carotene stored at different temperatures.

FIG. 6 shows clearly that the formulations are storage-stable. The amorphous morphology of the active ingredient does not change in the course of storage at different temperatures.

### EXAMPLE 3

### Production and Properties of the Composite Fibers Formed by PMMA and Epoxiconazole

In order to further illustrate the broad applicability of the method, composite fibers were produced from poly(methyl methacrylate) and epoxiconazole fungicide.

To produce composite fibers, polymer solutions were produced from poly(methyl methacrylate) Plexiglas® (PMMA) (Mw=430 000 g/mol, Tg=110° C. (Iso 11357)) and epoxiconazole fungicide (1-{[3-(2-chlorophenyl)-2-(4-fluorophenyl)oxiran-2-yl]methyl}-1H-1,2,4-triazole) in an ethanol/chloroform mixture (6:11) and spun to fibers. For this purpose, the solutions were spun with a syringe system under voltages between 40 and 45 kV.

The starting weights are listed in the following table:

| | | Active ingredient |
|---|---|---|
| | | content, % by |
| Material | Mass, g | weight |
| Ethanol | 5.53 | |
| Chloroform | 10.14 | |
| PMMA | 1.00 | |
| Epoxiconazole | 0.11 | 10 |
| ″ | 0.25 | 20 |
| ″ | 0.43 | 30 |
| ″ | 1.00 | 50 |

The concentration figures for the epoxiconazole active ingredient are based on total solids (PMMA+active ingredient). The concentration of the carrier polymer is based on total mass of solvent and polymer before the introduction of the active ingredient.

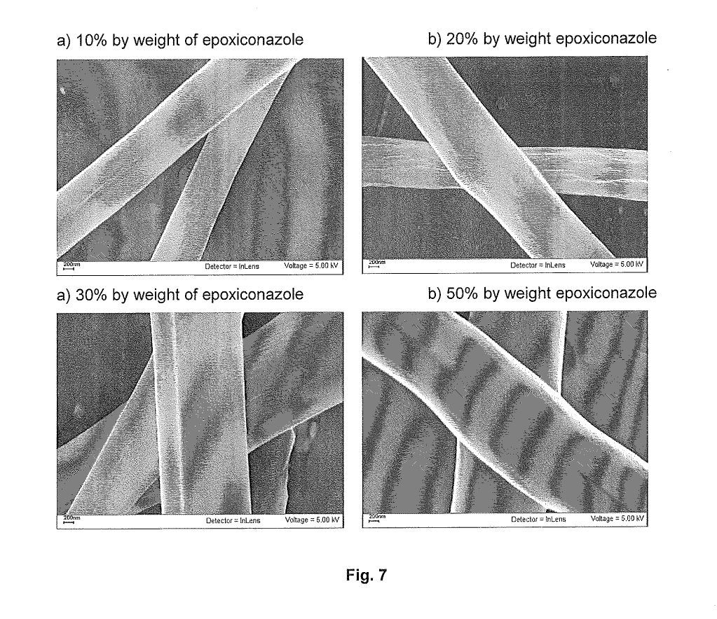

FIG. 7 shows the fiber morphology as a function of the active ingredient content.

The epoxiconazole active ingredient is present in the amorphous state in the fibrous sheetlike structures. This is shown by the wide-angle x-ray scattering analyses (WAXS), which were carried out with a Bruker D5005 diffractometer (monochromatized Cu-Kα radiation) in transmission. The samples were prepared on or between scotch tape.

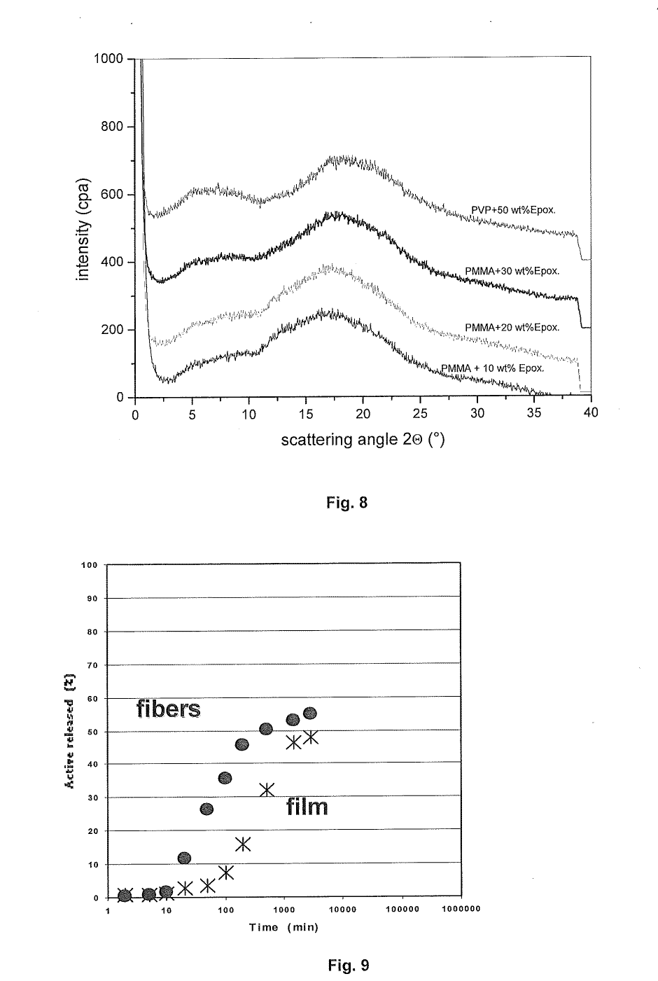

FIG. 8 shows the results of the WAXS analyses on fibrous sheetlike structures composed of PMMA-epoxiconazole.

### EXAMPLE 4

### Influence of the Specific Surface Area on Active Ingredient Release

The further advantage of the fibers is the high specific surface area thereof compared to films or other formulation forms. In order to demonstrate this, the release of the active ingredient from fibers and films was examined.

The fibrous sheetlike structures for the release tests were spun from a solution comprising 12% by weight of Ecoflex® (aliphatic-aromatic copolyester from BASE SE based on butanediol, adipic acid and terephthalic acid, Tg=−30° C., Tm=115° C., Mn=35 000; Mw=118 000; see also http://iwww.plasticsportal.com/products/ecoflex.html) (based on the total mass of the formulation before the introduction of the active ingredient), chloroform/i-propanol solvent mixture (95:5) and 10% by weight of epoxiconazole (based on solids (polymer+active ingredient)).

As a comparative sample for the fibrous sheetlike structure, the same polymer-active ingredient solution composed of 12% by weight of Ecoflex (based on the total mass of the formulation before the addition of the active ingredient) and 10% by weight of epoxiconazole (based on solids content) was painted onto a microscope slide, the solvent was evaporated and then a razor blade was used to remove the polymer/active ingredient film from the microscope slide.

The two samples were weighed into demineralized water in a concentration of 7 mg/l and stirred in a 0.5 I Erlenmeyer flask without interruption at constant speed on a magnetic stirrer. The measurement was effected by the method described above. The samples taken were analyzed for free active ingredient at a wavelength of 220 nm on an Agilent series 1100 HPLC system.

FIG. 9 shows the release profiles of epoxiconazole from biodegradable Ecoflex polyester as a film and as a fibrous sheetlike structure.

FIG. 9 shows that the release depends on the specific surface area of the carrier and can be controlled in this way.

### EXAMPLE 5

### Active Ingredient Release from Polymers with Different Solubility

The release can additionally be controlled via the solubility of the carrier polymer in the solvent. As an example, fibrous sheetlike structures were produced from polyvinylpyrrolidone, polymethyl methacrylate and Ecoflex with epoxiconazole, and the release in demineralized water was measured by the method described in example 4. The samples were prepared as follows:

a) 5% by weight of PVP, 20% by weight of epoxiconazole in ethanol-water mixture (9:1);
b) 12% by weight of Ecoflex, 20% by weight of epoxiconazole in chloroform-i-propanol mixture (95:5);
c) 6% by weight of PMMA, 20% by weight of epoxiconazole in chloroform-ethanol mixture (11:6).

The concentration figures for the epoxiconazole active ingredient are based on total solids (PVP+active ingredient). The concentration of the carrier polymer is based on total mass of solvent and polymer before the addition of the active ingredient.

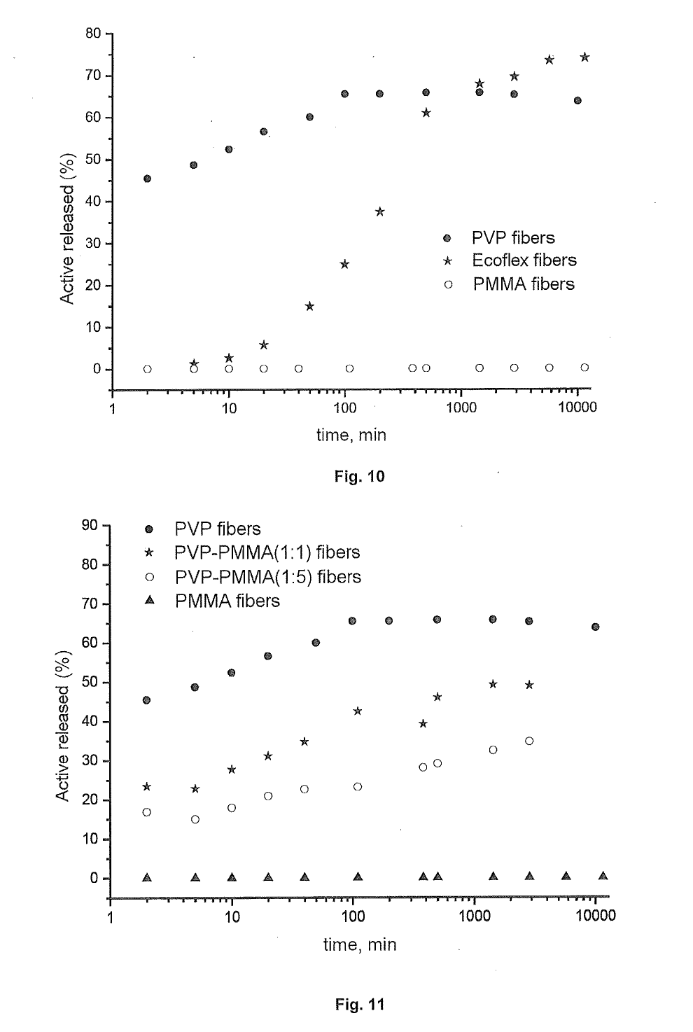

FIG. 10 shows the release profiles of epoxiconazole from biodegradable polyester Ecoflex, PVP and PMMA.

The water-soluble PVP releases epoxiconazole relatively rapidly. After only 2 min, about 40% of the epoxiconazole has escaped from the fibers. Epoxiconazole is released slowly from Ecoflex fibers in a retarded manner only after approximately 10 min. Only after one day has 40% of the active ingredient escaped from the fibers. Ecoflex is not water-soluble. The retarded and slow release could accordingly be attributable to diffusion of the epoxiconazole to the surface of the fibers or to partial degradation of the polyester. In contrast to PVP and Ecoflex fibers, no epoxiconazole is released from PMMA fibers in the first two days. PMMA fibers are not water-soluble, and the diffusion of the epoxiconazole out of the fibers into water is apparently also very slow or impossible.

### EXAMPLE 6

### Release from a Blend of Sparingly Miscible Polymers

The release profile can also be influenced via the polymer composition of fibrous sheetlike structures. For instance, it is possible to use carrier polymers of sparing or limited miscibility. The release of epoxiconazole from PVP and PMMA fibers and fibers of the blends thereof, PVP-PMMA(1:1) and PVP-PMMA(1:5), was tested using the following samples:

a) 5% by weight of PVP, 20% by weight of epoxiconazole in ethanol-water mixture (9:1);
b) 2.5% by weight of PVP, 2.5% by weight of PMMA, 20% by weight of epoxiconazole in chloroform-ethanol mixture (11:6);
c) 1% by weight of PVP, 5% by weight of PMMA, 20% by weight of epoxiconazole in chloroform-ethanol (11:6);
d) 6% by weight of PMMA, 20% by weight of epoxiconazole in chloroform-ethanol mixture (11:6).

The concentration figures for the epoxiconazole active ingredient are based on total solids (carrier polymer+active ingredient). The concentration of the carrier polymer is based on total mass of solvent and polymer before the introduction of the active ingredient.

FIG. 11 shows the release profile of epoxiconazole from fibrous sheetlike structures produced from PVP and blends thereof with PMMA.

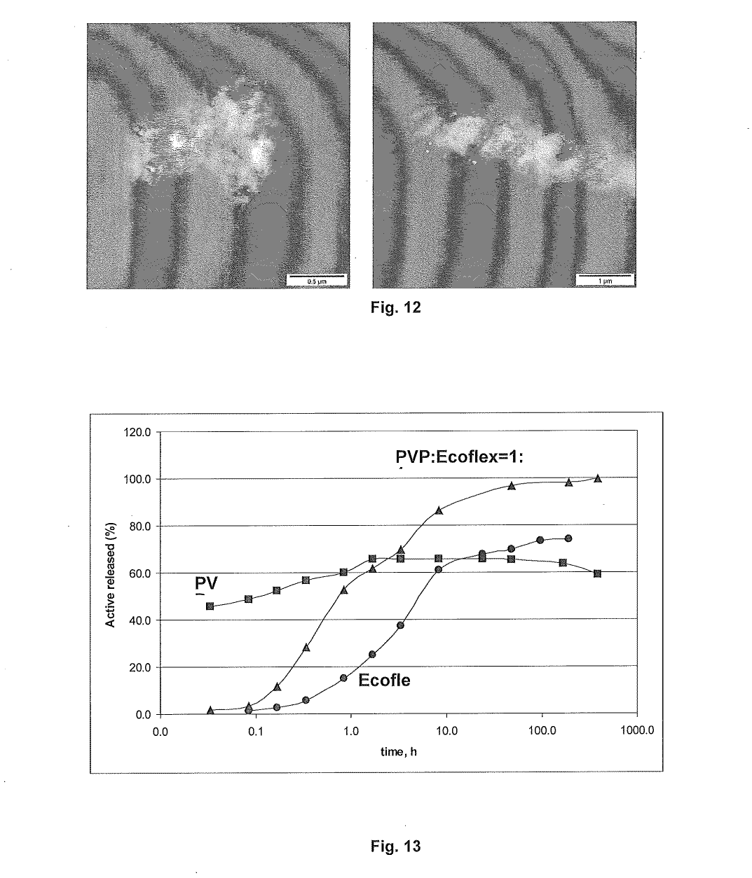

The release from the polymer blends corresponds very well to the expected behavior from the release profiles of the fibers of PVP or PMMA. For instance, the release decreases with rising PMMA content. The rapid initial release—the first measurement point is already well above 0%—is observed with the fibrous sheetlike structures. This behavior can be explained by the fact that the two carrier polymers are immiscible and form a structure in which high-PVP and low-PVP domains are present. This structuring is very clearly evident in TEM images. These show acrylate in a lighter color. FIG. 12 shows cross sections of the fibers of PMMA and PVP (5:1).

It is observed that the high-PVP phase is present preferentially at the fiber surface, whereas the acrylate phase is dominant in the interior. Rapid release can be explained by the dissolution of the high-PVP phase.

### EXAMPLE 7

### release from the Blend of Miscible Polymers

When miscible polymers are used, the result is fibrous sheetlike structures with homogeneous component distribution over the entire fibers. For the studies of the release profile, the following samples were employed:

a) 5% by weight of PVP, 20% by weight of epoxiconazole in ethanol-water mixture (9:1);
b) 4% by weight of PVP, 4% by weight of Ecoflex, 20% by weight of epoxiconazole in chloroform-i-propanol mixture (95:5);
c) 12% by weight of Ecoflex, 20% by weight of epoxiconazole in chloroform-i-propanol mixture (95:5).

The concentration figures for the epoxiconazole active ingredient are based on total solids (carrier polymer+active ingredient). The concentration of the carrier polymer is based on total mass of solvent and polymer before the introduction of the active ingredient.

FIG. 13 shows the release profiles of epoxiconazole from fibrous sheetlike structures produced from PVP and blends thereof with Ecoflex.

It is observed that the rapid active ingredient release, which is typical of PVP, is no longer present in the blend; the profile corresponds to the least soluble polymer, Ecoflex, and has become more rapid with time.

### EXAMPLE 8

### Production of the C16 Spider Silk Protein

The C16 spider silk protein was produced by biotechnological means using plasmid-containing Escherichia coli expression strains. The design and cloning of the C16 spider silk protein (also known as ADF4) are described in Hümmerich et al. (Biochemistry 43, 2004, 13604-13012). In contrast to the process described therein, C16 spider silk protein was produced in E. coli strain BL21 Gold (DE3) (Stratagene). It was grown in Techfors fermenters (Infors HAT, Switzerland) using a minimal medium and fed-batch techniques.

Minimal medium: 2.5 g/l citric acid monohydrate

4 g/l glycerol

12.5 g/l potassium dihydrogenphosphate

6.25 g/l ammonium sulfate

1.88 g/l magnesium sulfate heptahydrate

0.13 g/l calcium chloride dihydrate

15.5 ml/l trace element solution (40 g/l citric acid monohydrate;
11 g/l zinc(II) sulfate heptahydrate; 8.5 g/l diammonium iron(II) sulfate heptahydrate; 3 g/l manganese(II) sulfate monohydrate; 0.8 g/l copper(II) sulfate pentahydrate; 0.25 g/l cobalt(II) sulfate heptahydrate)

3 ml/l vitamin solution (6.3 mg/ml thiamine hydrochloride;
0.67 mg/ml, vitamin B12)

pH 6.3

Feed solution: 790 g/l glycerol

6.9 g/l citric acid monohydrate

13.6 g/l sodium sulfate

1.05 g/l diammonium iron(II) sulfate heptahydrate

13 mg/l thiamine hydrochloride

The cells were grown at 37° C. up to an OD₆₀₀ of 100, which was followed by the induction of protein expression with 0.1 mM isopropyl β-D-1-thiogalactopyranoside (IPTG). At the end of fermentation (8 to 12 hours after induction), the cultures were harvested. The main proportion of the protein was present in “inclusion bodies”.

After cell harvesting, the pellet was resuspended in 20 mM 3-(N-morpholino)propanesulfonic acid (MOPS), pH 7.0 (5L of buffer per kilogram of wet material). This was followed by cell disruption using an M-110EH microfluidizer (Microfluidics, US) at pressures of 1200 to 1300 bar. After sedimentation, the pellet after disruption comprised, as well as the inclusion bodies, also cell fragments and membrane constituents, which were removed by two wash steps. In a first wash step, the pellet was resuspended in 2.5 volumes of Tris buffer (50 mM Tris/HCl, 0.1% Triton X-100, pH 8.0) and then the remaining solids were sedimented by centrifugation. A second wash step was effected using Tris buffer (50 mM Tris/HCl, 5mM EDTA, pH 8.0). The pellet obtained once again after sedimentation was virtually free of membrane and cell fragments.

The cleaned inclusion bodies were dissolved in guanidinium thiocyanate (Roth, Germany), with addition of 1.6 g of guanidinium thiocyanate per 1 g of pellet (wet mass). The inclusion bodies dissolved while stirring with gentle heating (50° C.). To remove any insoluble constituents present, a centrifugation was subsequently carried out. In order to obtain an aqueous C16 spider silk protein solution, a 16-hour dialysis was then carried out against 5 mM potassium phosphate buffer (pH 8.0) (dilution factor of the dialysis: 200).

Contaminating E. coli proteins formed aggregates in the dialysis, which were removable by centrifugation. The protein solution obtained had a purity of ˜95% C16 spider silk protein.

The resulting aqueous protein solution can either be used directly for electrospinning or, for the purpose of better storability, processed further to protein microbeads. To produce C16 protein microbeads, the aqueous C16 spider silk protein solution is admixed with 0.25 volume of a 4 molar ammonium sulfate solution. Under the action of the ammonium sulfate, the protein monomers assemble to form spherical structures, which are referred to here as microbeads. The microbeads were removed by centrifugation, washed three times with distilled water and then freeze-dried.

### EXAMPLE 9

### Formulation of Clotrimazole as an Effect Substance by means of Electrospinning

In order to show the usability of the process described for the formulation of active substances, especially sparingly water-soluble active substances, the active pharmaceutical ingredient clotrimazole, by way of example, was encapsulated by means of electrospinning in sheetlike C16 spider silk protein structures.

For the production of a spinnable solution, C16 spider silk protein microbeads (14% [w/w]) and the active ingredient clotrimazole (10% [w/w]) were dissolved together in formic acid (98-100% p.a.). A beaker was initially charged with 200 ml of formic acid, and then 50.4 g of C16 spider silk protein and 36 g of clotrimazole (from Sigma, Germany) were stirred in gradually. Once the substances had dissolved completely, the solution was made up to 360 g with formic acid (98-100%).

Alternatively, it is also possible to use water-soluble C16 spider silk protein solution (see example 1) as the starting material basis. The active ingredient is then dissolved directly in the aqueous protein solution or, in the case of use of relatively high active ingredient concentrations, predissolved in an alternative solvent (e.g. formic acid) and then mixed with the protein solution. In order to increase the viscosity of the spinning solution, it is then additionally possible to add water-soluble polymers or polymer dispersions.

The solution of C16 spider silk protein and clotrimazole was spun in an Elmarco Nanospider apparatus for 3 hours. The voltage was 82 kV at an electrode distance of 18 cm. The temperature was 23° C. and the relative air humidity 35%. A serrated electrode was used for spinning. In order to achieve a sheetlike protein structure of maximum thickness, the carrier nonwoven was left stationary. Alternatively, the carrier nonwoven can also be moved with an advance rate to achieve thinner sheetlike protein structure layers in a defined manner. The protein fibers obtained from the batch were subsequently dried at 40° C. under reduced pressure overnight.

The electron microscopy analysis of the thus produced sheetlike C16 spider silk protein structures with incorporated clotrimazole showed that the structures are principally fibers having a diameter about 50 nm up to 1 μm (FIG. 14).

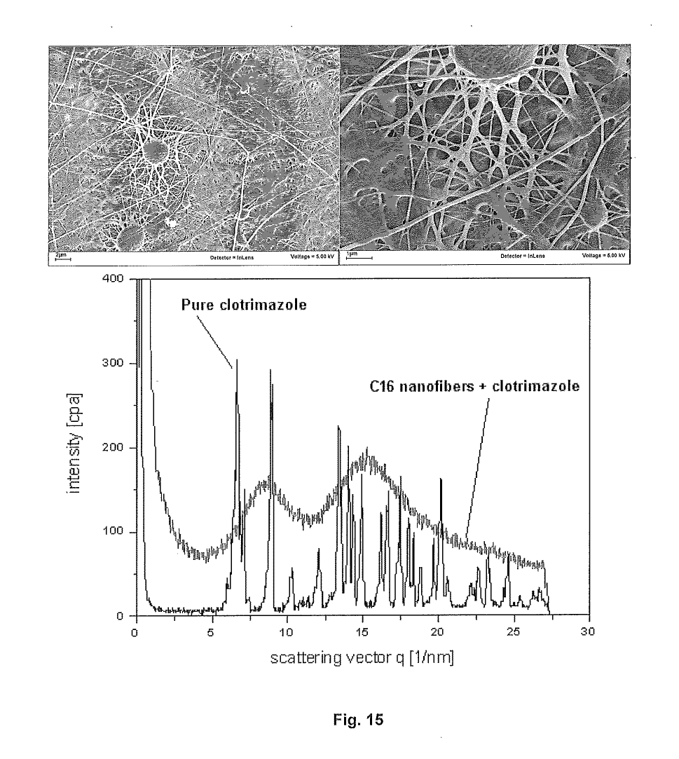

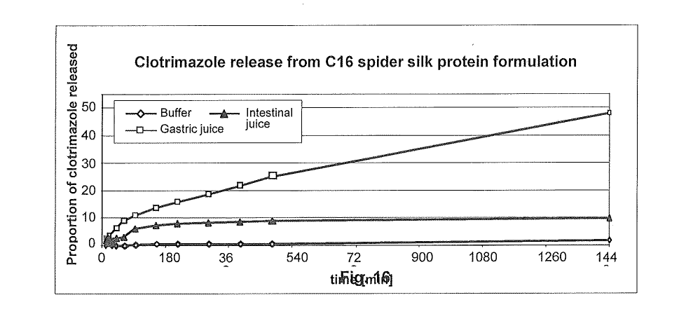

In contrast to pure clotrimazole, x-ray diffraction does not show any crystalline peaks in the C16 spider silk protein/clotrimazole formulation (FIG. 15). Accordingly, it can be assumed that the active ingredient has been encapsulated in amorphous form or as a solid solution, which can positively influence the bioavailability thereof.

In order to test active ingredient release from a very relevant administration form, the sheetlike C16 spider silk protein structures were used to press tablets. In each case 300 mg of material were pressed under reduced pressure and at pressure 100 bar in a KBr press (from Paul-Otto-Weber, Germany) for approx. 10 min. The tablets had a diameter of about 13 mm and a thickness of about 2 mm.

The release of clotrimazole from the tablets was tested in two different tests. Synthetic gastric juice (0.1 g of NaCl; 0.16 g of pepsin; make up 0.35 ml of HCl to 50 ml, pH 1-2) and synthetic intestinal juice (dissolve 3.4 g of KH₂PO₄ in 12.5 ml of water+make up 3.85 ml of 0.2N NaOH to 25 ml+make up 0.5 g of pancreatin to 50 ml, pH 6.8) were used to simulate the release of active ingredient under proteolytically active conditions in the digestive tract. A further test was effected in 5 mM potassium phosphate buffer (pH 8.0), and only a small release of active ingredient should be observed under these control conditions. 20 ml of the particular digestive juice or buffer were added per tablet, and the mixtures were incubated with slight agitation at 37° C. and 80 rpm. The clotrimazole released was quantified on the basis of its poor water solubility (and hence tendency to form aggregates in aqueous systems) after extraction of the supernatant with THF by absorption photometry determination at 262 nm (3 ml of supernatant+3 ml of THF+spatula-tip of NaCl, vortex vigorously, 1 min at 15 000×g, analyze upper phase, dilute if appropriate).

While only a maximum of 2% of the amount of active ingredient encapsulated was released in the control experiment (buffer without proteases), about 50% release is achieved within 24 h in gastric juice, controlled by the enzymatic activity present (proteases) (FIG. 16). In the course of this, the clotrimazole active ingredient is released continuously. In intestinal juice, in contrast, only about 20% of the active ingredient is released after 24 h (FIG. 16). The C16 spider silk protein/clotrimazole formulation appears to be so stable at the comparatively neutral pH values which exist therein over the time range in question that only attenuated release is observed.

In order to determine the proportion of clotrimazole as yet unreleased from the formulation after 24 h, the mixtures comprising the proteolytically undegraded C16 spider silk protein fibers were admixed with 3 ml of tetrahydrofuran (THF) and incubated with shaking for a further max. 48 h. Subsequently, the active ingredient content was quantified by absorption photometry at 262 nm. It was thus possible to use the end value and the previously determined intermediate values to determine the loading density of the C16 spider silk protein formulation with the clotrimazole active ingredient. The loading density for all tablets examined was between 27% and 33% [w/w], which gave an average loading density of the sheetlike C16 spider silk protein structure pressed to tablets with about 30% [w/w] clotrimazole (see table below).

Loading densities of the C16 spider silk protein formulation
(tablets) with the clotrimazole active ingredient.

| | | | mg of | |
|---|---|---|---|---|
| | Tablet | Clotrimazole | clotrimazole | Loading |
| | mass | in solution | per | density |
| Experiment | [mg] | [mg] | mg of tablet | [%] |
| Buffer | 304 | 92.2 | 0.303 | 30.3 |
| Gastric juice | 302 | 99.1 | 0.328 | 32.8 |
| Intestinal juice | 299 | 82.0 | 0.274 | 27.4 |

| | |
|---|---|
| Average loading density | 30.2 |

Reference is made explicitly to the disclosure of the publications cited herein.

1.-25. (canceled)

26. An active ingredient-containing fibrous sheetlike structure comprising a fibrous, polymeric, soluble and/or degradable active ingredient carrier and at least one low or high molecular weight active ingredient which is associated with the carrier and can be released by the fibrous sheetlike structure, wherein the carrier is a composite polymer which comprises a mixture of at least two polymer components, wherein these at least two polymer components differ in at least one property which is selected from
a) solubility in solvents,
b) molecular weight
c) glass transition temperature/melting point; and
d) degradability;
wherein the composite polymer is selected from
i) mixtures of at least 2 miscible synthetic homo- or copolymers; and
ii) mixtures of at least 2 immiscible synthetic homo- or copolymers; and wherein the polymers are selected from:
polyvinyl acetals, polyvinyl esters, polyamides, polyesters, polyacrylamides, polymethacrylamides, polyhydroxybutyrates, polyvinyl alcohols, acetylated polyvinyl alcohols, polyvinylformamides, polyvinylamines, polycarboxylic acids, polyacrylamides, poly(2-hydroxyethyl acrylates), poly(N-isopropylacrylamides), polymethacrylamides, poly-N-vinylpyrrolidones, poly(ethyleneimines), polyacrylates, polymethacrylates, polystyrenes, copolymers based on styrene, N-vinylpyrrolidone copolymers, polycaprolactones and poly(N-vinylcaprolactams).

27. The fibrous sheetlike structure according to claim 26, wherein the polymers are selected from poly-N-vinylpyrrolidones, polymethyl methacrylates, acrylate-styrene copolymers, polyvinyl alcohols, polyvinyl acetates, polyamides and polyesters.

28. The fibrous sheetlike structure according to claim 26, wherein the at least one active ingredient is in amorphous, semicrystalline or crystalline form.

29. The fibrous sheetlike structure according to claim 26, wherein the active ingredient is integrated into and/or absorbed onto the carrier.

30. The fibrous sheetlike structure according to claim 26, wherein the fibrous, active ingredient-containing carrier is obtainable by a spinning process.

31. The fibrous sheetlike structure according to claim 30, wherein the fibrous, active ingredient-containing carrier is obtainable by an electrospinning process with an electrospinnable solution comprising, in each case in dissolved form, the at least one active ingredient and the mixture of at least two polymer components.

32. The fibrous sheetlike structure according to claim 26, wherein the polymer components each independently have molar masses in the range from about 500 to 10 000 000.

33. The fibrous sheetlike structure according to claim 26, wherein the diameter of the active ingredient carrier fibers is 10 nm to 100 μm.

34. The fibrous sheetlike structure according to claim 26, wherein the diameter of the active ingredient carrier fibers is 100 nm to 2 μm.

35. The fibrous sheetlike structure according to claim 26, wherein the active ingredient loading is about 0.1 to 80% by weight, based on the solids content of the fibrous sheetlike structure.

36. The fibrous sheetlike structure according to claim 26, selected from polymer fibers and polymer nonwovens.

37. The fibrous sheetlike structure according to claim 26, wherein the active ingredient is present in the fibers in molecular dispersion or in nanoparticulate dispersion.

38. An active ingredient-containing formulation comprising the fibrous sheetlike structure according to claim 26 in processed form, optionally in combination with at least one further formulating assistant.

39. The formulation according to claim 38, comprising the fibrous sheetlike structure in comminuted or uncomminuted form.

40. The formulation according to claim 38, comprising the fibrous sheetlike structure in compacted form, in powder form or applied to a carrier substrate.

41. The formulation according to claim 38, selected from cosmetic formulations, human and animal pharmaceutical formulations, agrochemical formulations, food and animal feed additives.

42. A process for production of an active ingredient-containing formulation which comprises utilizing the active ingredient-containing fibrous sheetlike structure according to claim 26.

43. A method for controlled release of an active ingredient comprising utilizing the formulation according to claim 37.

44. A process for producing a fibrous sheetlike structure according to claim 26, which comprises
a) mixing the at least one active ingredient together with the carrier polymer components in a combined liquid phase and
b) then embedding the active ingredient into a polymeric composite fiber is performed by means of spinning processes.

45. The process according to claim 44, wherein the at least one active ingredient and the polymer components are mixed in a solvent phase and spun from this mixture.

46. The process according to claim 44, wherein the at least one active ingredient and the polymer components are mixed in a mixture of at least two mutually miscible solvents, wherein active ingredients and polymers are soluble at least in one of the solvents, and spun from this mixture.

47. The process according to claim 44, wherein the spinning process is an electrospinning process or a centrifuge spinning process.

48. The process according to claim 44, wherein the operating temperature is in the range from about 0 to 90° C.

49. The fibrous sheetlike structure according to claim 26, which is essentially free of low molecular weight active ingredients.

50. A process for production of an active ingredient-containing formulation which comprises utilizing fibrous sheetlike structure according to claim 49.

51. The process according to claim 50, wherein the formulation is selected from cosmetic formulations, human and animal pharmaceutical formulations, agrochemical formulations, food and animal feed additives.
